(19) United States
(12) Patent Application Publication
(10) Pub. No.: US 2025/0244327 A1
(43) Pub. Date: Jul. 31, 2025

(54) DEVICES, SYSTEMS, AND METHODS FOR ANALYZING NANOPARTICLES AND MICROPARTICLES

(71) Applicant: PURDUE RESEARCH FOUNDATION, West Lafayette, IN (US)

(72) Inventors: Arezoo Ardekani, West Lafayette, IN (US); Mohit Verma, West Lafayette, IN (US); Madhi Hosseini, West Lafayette, IN (US); Murali Maruthamuthu, Chapel Hill, NC (US); Ehsan Esmaili, West Lafayette, IN (US); Tae Hong Kim, West Lafayette, IN (US); Shreya Milind Athalye, West Lafayette, IN (US); Miad Boodaghizaji, West Lafayette, IN (US)

(21) Appl. No.: 18/854,363

(22) PCT Filed: Apr. 12, 2023
(86) PCT No.: PCT/US23/18303

§ 371 Date: Oct. 4, 2024

Related U.S. Application Data
(60) Provisional application No. 63/330,104, filed on Apr. 12, 2022.
(60) Provisional application No. 63/356,825, filed on Jun. 29, 2022.

(51) Int. Cl. G01N 33/569; B01L 3/00; G01N 15/075

(52) U.S. Cl. CPC G01N 33/56983; B01L 3/502761; G01N 15/075; B01L 2200/0647; B01L 2300/0663; B01L 2300/0838; B01L 2300/168; B01L 2400/0436

(57) ABSTRACT

Devices, systems, and methods for analyzing nanoparticles and microparticles. An exemplary device for analyzing microparticles and nanoparticles is disclosed herein, comprising an elongated capillary tube having a square cross-section, and a substrate configured to receive the elongated capillary tube thereon, wherein the elongated capillary tube is affixed to the substrate using an adhesive.

### PRIORITY

The present application is related to, and claims the priority benefit of, U.S. provisional patent application Ser. No. 63/356,825, filed Jun. 29, 2022, and U.S. provisional patent application Ser. No. 63/330,104, filed Apr. 12, 2022, the contents of which are incorporated herein directly and by reference in their entirety.

### BACKGROUND

The recent outbreak of COVID-19 proved the importance of robust anti-viral medications to stop the spread of pandemic viral infections. Anti-viral drugs and vaccines are the two major solutions to keep viral infections at bay. A recent study suggested that, for example, the COVID-19 vaccine saved approximately 20 million human lives in one year. Measles, mumps, rubella, and varicella (MMRV) are common viral childhood diseases that can have serious complications. Developing efficient methods to mass produce MMRV paves the way for limiting the spread of the MMRV globally. The vaccine development flourished in the early 20th century, and Maurice Hilleman at Merck & Co., Inc., a pioneer in the development of vaccinations, developed Rubeovax™ in 1968, the first commercial live vaccine for measles. Vaccine development and production have been continuously improving in upstream and downstream processing. Vaccine production involves challenging processes such as viral vector development, effective purification, polishing steps, and formulation with stable storage conditions. These processes require comprehensive and continuous quality management to maintain the product's efficacy and ensure public safety. With the advancement in viral vector-driven gene therapies and vaccine production, there is a growing interest in improving the continuous production of virus-like particle (VLP)-based vaccines. The development of continuous manufacturing processes in the vaccine industry demands rapid, robust, and continuous analytical methods (process analytical technology (PAT) tools) to understand real-time manufacturing processes.

Viral epidemics and pandemics require rapid development and manufacturing of vaccines to limit the spread of the disease and minimize loss of life. Rapid production of vaccines requires standardized processes for controlling the quality of the product, demanding more innovative and reliable methods in PAT.

An increase in the demand for vaccination and gene therapy has augmented the need for developing an efficient platform for large-scale manufacturing using continuous approaches in combination with VLP production (for example, lipid nanoparticles and adenoviruses). Viral-based protein production in perfusion mode has already been explored for intensified production. This technology clearly shows advantages, such as higher titer compared with batch processes. Complete continuous manufacturing of a viral vaccine needs further advancements in analytical technology to guarantee product quality.

With the advancement in viral vector-driven gene therapies and vaccine production, there is an increasing interest in optimizing the continuous production of virus-like particle (VLP)-based vaccines. Continuous monitoring of viral particles during vaccine manufacturing is challenging due to their small size. To overcome this limitation, a robust system that can be non-destructive and provide a relative quantification of viral load during the process is needed.

Non-invasive in-line sensors such as Raman probes (Raman spectroscopy) hold great potential due to their higher sensitivity to read the molecular fingerprints of chemical and biological molecules, species, or products. Raman spectra possess clear spectral features that can be easily assigned to different chemical compounds. Additionally, minimal sample preparation is sufficient for making accurate quantitative predictions using Raman spectra. In other words, Raman spectroscopy provides invaluable information for various analyte molecules even in ultra-low concentrations. Similarly, absorption spectroscopy is a robust technique that, owing to high sensitivity and large signal-to-noise ratio, has the potential to be implemented as a great tool to make predictions. Generally, both Raman and absorption spectra have been widely used for particle detection and identification and quantitative analysis.

Recently, machine learning (ML) has become popular for making predictions based on spectroscopy data. Both supervised and unsupervised ML techniques have been applied to Raman signals to make predictions. Particularly, Raman spectroscopy has been utilized for cancer predictions. For instance, techniques, such as the principal component analysis or artificial neural networks have been utilized for detecting cervical cancer. Furthermore, Raman signals have been utilized for classification problems, such as classifying bacteria, viral infections, and fungal infections. Additionally, Raman spectroscopy has been applied for regression purposes, such as predicting the concentration of the markers of interest, such as sensing the pH and lactate in body fluids. Absorption spectroscopy also has been utilized for classification purposes, such as characterization of proteins classification of wines, quantifying the concentration of organic acids. Furthermore, the joint Raman and absorption spectra have been applied to predict the values of concentrations.

Previous studies, in particular, have confirmed the capability of ML techniques in making quantitative predictions based on Raman or absorption signals. However, a comparison of these signals and their strength in making accurate ML-based predictions for viral samples, such as MMRV has not been studied before.

The main challenge in the continuous manufacturing of viral vaccines is the lack of PAT tools for monitoring of product quality. Analytical techniques used for the quantification and characterization of viral particles build upon quantitative polymerase chain reaction (qPCR), droplet digital polymerase chain reaction (ddPCR), dynamic light scattering (DLS), flow virometry, and quantitative capillary western blot. However, these techniques are currently employed as at-line or off-line methods. Therefore, improvement is still needed to ensure precise real-time, in situ monitoring of the quality and quantity of the viral particles. As described herein, the present disclosure includes disclosure of the development of a Raman spectroscopy-based system that can monitor the quality and quantity of the VLPs in flow with a simple quartz capillary tube setup as well as coupled with an acoustic setup to focus VLPs. ML techniques are then used to process the data.

### BRIEF SUMMARY

The present disclosure includes disclosure of a Raman spectroscopy-based tool to characterize attenuated human cytomegalovirus (CMV) at a concentration between 4.5×10⁹ particles/mL to 2.9×10¹¹ particles/mL at flow rates between 50 μL/min to 250 μL/min within a square quartz capillary tube. This process analytical technology (PAT) enables continuous monitoring of viral particles in a continuous bioprocess manufacturing setup for vaccine production. Using Western blotting and dynamic light scattering, the present disclosure demonstrates that the Raman laser is non-invasive, given that the samples maintain their integrity before and after exposure, which is believed to be the first disclosure on characterizing CMV particles using Raman spectroscopy. This technology can also be extended to study the quality of the viral particles in flow for continuous manufacturing of biologics and vaccines.

As discussed in further detail herein, the present disclosure includes disclosure of the use of Raman spectroscopy as a PAT tool, combined with machine learning algorithms, to characterize viral particles for continuous in-process monitoring of vaccine contents. Acoustic methods are used to improve the Raman signal and establish the basis for a standardized detection method. Different virus-like particles and vaccines are tested and characterized, including inactivated SARS-COV-2 and influenza vaccine, showing this new capability to identify the viral particles using the Raman spectra and quantifying them at different stages of production.

Furthermore, a fully trained and validated deep learning software package has been developed to analyze viral particles. The present disclosure includes disclosure of a newly designed double convolutional neural network (CNN) that uses Raman and absorbance spectra as the inputs and predicts the concentration of the samples as the output. CNNs are a subset of deep learning methods that can automatically capture important parts of the input signals and learn hidden features that are hard to detect by human eyes. The incorporation of artificial intelligence and machine learning deepens the overall understanding of the fundamental science underlying vaccine processes, especially for real time, in situ analysis. The spectro-acoustic PAT of the present disclosure enables the real-time quantification of virus particles necessary for the continuous manufacturing of vaccines.

The present disclosure includes disclosure of a device for analyzing microparticles and nanoparticles, comprising an elongated capillary tube having a square cross-section, and a substrate configured to receive the elongated capillary tube thereon, wherein the elongated capillary tube is affixed to the substrate using an adhesive.

The present disclosure includes disclosure of a device, further comprising a first plastic tube positioned relative to and about the first end of the elongated capillary tube and coupled thereto using the adhesive.

The present disclosure includes disclosure of a device, further comprising a second plastic tube positioned relative to and about a second end of the elongated capillary tube and coupled thereto using the adhesive.

The present disclosure includes disclosure of a device, further comprising a pump in fluid communication with one of the first plastic tube and the second plastic tube, the pump configured to pump a fluid having a virus therein through the first plastic tube or the second plastic tube and into the elongated capillary tube.

The present disclosure includes disclosure of a device, wherein the substrate comprises a glass slide.

The present disclosure includes disclosure of a device, wherein the substrate comprises a lithium niobate (LiNBO₃) substrate.

The present disclosure includes disclosure of a device, wherein the substrate comprises an interdigital transducer (IDT) electrode positioned thereon or defined therein.

The present disclosure includes disclosure of a device, wherein a surface acoustic wave (SAW) is activated by a radio signal from a power supply connected to the IDT.

The present disclosure includes disclosure of a device, wherein the SAW produced by the IDT travels through the LiNBO₃ substrate and a thin layer of UV epoxy to reach the elongated capillary tube.

The present disclosure includes disclosure of a device, wherein the SAW causes the bottom of the elongated capillary tube to vibrate, which generates an acoustic force that acts on the fluid and microparticles and nanoparticles inside.

The present disclosure includes disclosure of a device, wherein the adhesive comprises an ultraviolet (UV) epoxy resin.

The present disclosure includes disclosure of a device, prepared by applying a photoresist onto the LiNBO₃ substrate, applying ultraviolet light to a portion of the photoresist, developing the photoresist to form a void therein, and depositing metal onto the photoresist and into the void produced by the step of developing the photoresist.

The present disclosure includes disclosure of a device, further prepared by removing the photoresist from the LiNBO₃ substrate, effectively forming an interdigital transducer (IDT), and applying a conducting epoxy to the metal deposited onto the LiNBO₃ substrate and applying a clear ultraviolet (UV) epoxy to a portion of the LiNBO₃ substrate.

The present disclosure includes disclosure of a device, further prepared by affixing the elongated capillary tube to the LiNBO₃ substrate upon the UV epoxy.

The present disclosure includes disclosure of a device, wherein the deposited metal comprises titanium and gold.

The present disclosure includes disclosure of a device, wherein the titanium is deposited onto the LiNBO₃ substrate, and wherein the gold is deposited onto the titanium.

The present disclosure includes disclosure of a device, wherein the elongated capillary tube has a square width dimension selected from the group consisting of 400 μm, 600 μm, and 800 μm.

The present disclosure includes disclosure of a device, wherein the elongated capillary tube has a thickness dimension selected from the group consisting of 100 μm, 150 μm, and 200 μm.

The present disclosure includes disclosure of a device, wherein the IDT comprises two opposing vertical bars, each vertical bar of the opposing vertical bars having alternating horizontal fingers extending therefrom.

The present disclosure includes disclosure of a device, wherein the IDT is configured to provide an operating frequency ranging from 1.7 MHz to 4.6 MHz when is activated by the radio signal from the power supply connected to the IDT.

The present disclosure includes disclosure of a device, wherein spaces exist between each of the alternating horizontal fingers, the spaces being at or about 2 μm in depth.

The present disclosure includes disclosure of a device, forming part of a system, the system further comprising a function generator in communication with a power supply, the power supply effectively coupled to the IDT, and a Raman analyzer positioned relative to the elongated capillary tube.

The present disclosure includes disclosure of a system, configured so that wherein when power is supplied from the power supply to the IDT, the IDT is configured to generate a surface acoustic wave (SAW) that travels through the LiNBO₃ substrate to the elongated capillary tube, causing the elongated capillary tube to vibrate.

The present disclosure includes disclosure of a system, configured so that the vibration of the elongated capillary tube causes particles suspended within a fluid within the elongated capillary tube to move due to an acoustic force acting thereupon.

The present disclosure includes disclosure of a system, configured so that movement of the particles can be detected by the Raman analyzer.

The present disclosure includes disclosure of a system for analyzing microparticles and nanoparticles, comprising an exemplary device of the present disclosure, and a Raman microscope having a lens directed at the device, wherein when the fluid having the virus therein is pumped into the elongated capillary tube, the Raman microscope is used in connection with an excitation laser to obtain Raman spectra in connection with the virus.

The present disclosure includes disclosure of a system for analyzing microparticles and nanoparticles, comprising an exemplary device of the present disclosure, and a generator configured to generate an electrical signal for actuation with different frequencies, and an amplifier configured to amplify the electrical signal.

The present disclosure includes disclosure of a system, wherein when the amplified electrical signal is applied to the IDT, the device vibrates in response.

The present disclosure includes disclosure of a system, further comprising a stainless-steel plate positioned below the device, the stainless-steel plate configured to eliminate the interference of Raman signals beneath the device so that the Raman signal contains only signals from the stainless-steel, the UV epoxy, the elongated capillary tube, and LiNBO₃.

The present disclosure includes disclosure of a system, wherein Raman measurements are obtained under laser power directed toward a sample present within the elongated capillary tube.

The present disclosure includes disclosure of a method for analyzing microparticles and nanoparticles, comprising the steps of positioning an exemplary device of the present disclosure relative to a Raman spectroscopy device, and operating the Raman spectroscopy device to obtain data relative to the virus present within the capillary tube.

The present disclosure includes disclosure of a method for analyzing microparticles and nanoparticles, comprising the steps of positioning an exemplary device of the present disclosure relative to a Raman spectroscopy device, and operating a generator coupled to an amplifier and configured to generate an electrical signal upon the IDT, wherein a surface acoustic wave (SAW) generated by the IDT due to the electrical signal travels through the LiNBO₃ substrate and a thin layer of UV epoxy to reach the elongated capillary tube.

The present disclosure includes disclosure of a method, wherein the SAW causes the bottom of the elongated capillary tube to vibrate, which generates an acoustic force that acts on the fluid and microparticles and nanoparticles inside.

The present disclosure includes disclosure of a method, further comprising the step of operating the Raman spectroscopy device to obtain data relative to the microparticles and nanoparticles within the elongated capillary tube.

The present disclosure includes disclosure of a method, further comprising the steps of processing the Raman and absorption spectra to analyze viral samples.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a schematic of an exemplary system and elements used therein, with subsection (a) depicting samples of CMV viral dilutions from 2.9×10¹¹ (particles/mL) to 4.5×10⁹ (particles/mL), subsection (b) showing a square quartz capillary tube with an inlet and an outlet for flow, and subsection (c) showing Raman imaging with the viral flow using inVia™ confocal microscope, with an inset showing at-line Raman data collection, according to exemplary embodiments of the present disclosure.

FIG. 2 shows Raman spectroscopic analysis for attenuated cytomegalovirus (CMV) viral particles in static conditions with different dilutions on the stainless-steel substrate, with subsection (a) showing Raman spectra for all the dilutions in the static condition C₁=2.90×10¹¹, C₂=1.45×10¹¹, C₃=7.25×10¹⁰, C₄=3.62×10¹⁰, C₅=1.81×10¹⁰, and C₆=8.96×10⁹ (each in particles/mL), and subsection (b) showing the area under the curve for 2900 cm⁻¹ peak of different viral dilutions, according to exemplary embodiments of the present disclosure.

FIG. 3 shows Raman spectra for CMV viral particles with different dilutions and different flow rates, with subsection (a) showing 2.90×10¹¹ particles/mL, subsection (b) showing 1.45×10¹¹ particles/mL, subsection (c) showing 7.25×10¹⁰ particles/mL, subsection (d) showing 3.62×10¹⁰ particles/mL, subsection (e) showing 1.81×10¹⁰ particles/mL, subsection (f) showing 8.96×10⁹ particles/mL, and subsection (g) showing 4.5×10⁹ particles/mL, according to exemplary embodiments of the present disclosure.

FIG. 4 shows the area under the curve at approximately 2900 cm⁻¹ at different flow rates in the capillary tube setup. Raman analysis with attenuated cytomegalovirus (CMV) viral particles in the fluid flow setup with viral dilutions (4.5×10⁹ to 2.90×10¹¹ particles/mL) and fluid flow (50 μl/min to 250 82 l/min), according to exemplary embodiments of the present disclosure.

FIG. 5 shows in subsection (a) DLS analysis for the lowest (4.5×10⁹ particles/mL), and in subsection (b) DLS analysis for the highest concentration (2.90×10¹¹ particles/mL) viral concentrations (noting that the laser is not damaging the viral particles), according to exemplary embodiments of the present disclosure. The experiments were conducted on 3 different days with different samples.

FIG. 6 shows the functional analysis of the viral particles before and after laser exposure, with subsection (a) showing the SDS page before and after laser exposure (lane 3: before laser exposure and lane 4: after laser exposure), and with subsection (b) and subsection (c) each showing Western blot analysis and electrogram respectively for the samples, according to exemplary embodiments of the present disclosure.

FIG. 7 shows a double convolutional neural network (CNN) that uses Raman and turbidity (absorbance) spectrums as the inputs and predicts the concentration of the samples as the output, according to an exemplary embodiment of the present disclosure.

FIG. 8 shows iteration data (subsection (a)) used to generate prediction data (subsection (b)) with an R² above 90% for all cases, according to an exemplary embodiment of the present disclosure.

FIG. 9 shows the comparison between measured Raman signals and predicted values (Equations S1 and S2), with subsection (a) having no correction applied (b=0), and with subsection (b) having correction applied (b=0.42 cm), whereby the insets show zoomed-in area for low Raman intensity signals, according to exemplary embodiments of the present disclosure.

FIG. 10 shows the velocity and shear stress calculations in the square capillary tube, with subsection (a) showing a velocity contour in the x-y plane, subsection (b) showing a velocity profile (v_{z}) in the middle of the capillary in the z direction, subsection (c) showing shear stress in the x-y plane, and subsection (d) showing shear stress in x-z plane, according to exemplary embodiments of the present disclosure.

FIG. 11 shows Raman signals for different flow rates, with subsection (a) showing signals for the concentration of 2.9×10¹¹ (particles/mL), and with subsection (b) showing signals for the concentration of 1.8×10¹¹ (particles/mL), according to exemplary embodiments of the present disclosure.

FIG. 12 shows zoomed Raman signals at 2900 cm⁻¹ for the low concentrations at different flow rates, with subsection (a) showing 1.81×10¹⁰ particles/mL, subsection (b) showing 8.9×10⁹ particles/mL, and subsection (c) showing 4.5×10⁹ particles/mL), according to exemplary embodiments of the present disclosure.

FIG. 13 shows (a) Nanoparticles in the capillary tube with the acoustic device off and on (subsection (a)), and the light intensity in channel cross sections increases (subsection (b)), whereby the intensity for the device on is 129 and off is 20.6±2.95 (increased by factor: 129/20.6˜6.2), according to exemplary embodiments of the present disclosure.

FIG. 14 shows a schematic of droplet spinning between two IDTs (subsection (a)), and that the light intensity in droplets increases and 1 μm and 500 nm particles get enriched at the center of droplets (subsection (b)), with f=23.8 MHz and volume=13 & 10 μl, according to exemplary embodiments of the present disclosure.

FIG. 15 shows in subsection (a) a PDMS channel bonded on top of the device between to IDTs, in subsection (b) particle enrichment for 1 μm, and in subsection (c) 500 nm particles at the center middle of the channel, with f=14.353 MHZ, according to exemplary embodiments of the present disclosure.

FIG. 16 shows Raman spectra for four different vaccines and their buffers, namely NR-31798 Alfuria (subsection (a)), Flucelvax (subsection (b)), NR-51702 Fluzone (subsection (c)), and IMOVAX® RABIES (subsection (d)), according to exemplary embodiments of the present disclosure.

FIG. 17 shows data comparing the developed algorithms versus obtained values for different viral concentrations, with absorption data shown at the top and Raman data shown at the bottom, according to exemplary embodiments of the present disclosure.

FIG. 18A shows a schematic of a device used in connection with a system, according to exemplary embodiments of the present disclosure.

FIG. 18B shows a device comprising a capillary tube, two additional flow tubes, and a pump, according to an exemplary embodiment of the present disclosure.

FIG. 19 shows a device comprising a capillary tube, two additional flow tubes, a pump, and a transducer, according to an exemplary embodiment of the present disclosure.

FIG. 20 shows a chronologically ordered schematic illustration of the fabrication steps, whereby subsection (a) shows that the process uses a lithium niobate wafer, subsection (b) shows the spin-coated photoresist onto the LiNBO₃ wafer, subsection (c) shows the maskless photolithography for the patterning, subsection (d) shows developing the photoresist, subsection (e) shows metal deposition, subsection (f) shows lifting the photoresist off, subsection (g) shows the application of epoxies (whereby conducting epoxy is applied to the electrode, while clear UV epoxy attaches the glass capillary to the wafer), and subsection (h) shows the conclusion of fabrication with the bonding of the capillary to the wafer, according to exemplary embodiments of the present disclosure.

FIG. 21 shows time-lapse images and intensity of 500 nm fluorescent particles under the acoustic field, with subsection (a) and subsection (b) showing time-lapse images of the beads' motion in 400 μm and 800 μm capillaries, respectively (whereby the scale bar indicates 200 μm. and 400 μm, respectively), with subsection (c) showing the Gaussian fitting of the intensity at different times in the 800 μm capillary, and with subsection (d) showing the intensity of the peak concentration (i.e., Gaussian curve's maximum point) as a time function, according to exemplary embodiments of the present disclosure.

FIG. 22 shows the Raman signals obtained from capillaries of different sizes and polystyrene beads, with subsection (a) and subsection (b) showing the Raman signals obtained from 800 μm and 400 μm capillaries, respectively, bonded on LiNBO₃ wafer, with subsection (c) showing a comparison of Raman intensity between capillaries of various dimensions, with subsection (d) showing Raman intensity at different magnifications, and with subsection (e) showing the Raman intensity obtained from polystyrene beads and epoxy on LiNBO₃ was normalized by the area under the curve, according to exemplary embodiments of the present disclosure.

FIG. 23 shows Raman measurements on the focused region in solutions of various concentrations, with subsection (a) showing a normalized Raman measurement of the focused region of 1.44 E+10 particle/mL (whereby the solid line measures the concentrated region, while the dashed line corresponds to the device off), and subsections (b), (c), (d), (e), (f), (g), and (h) showing subtraction of the normalized Raman intensity when the device off from normalized Raman intensity from the focused region, where the initial input concentration was 1.44 E+10, 7.21 E+8, 1.44 E+8, 7.21 E+7, 1.44 E+7, 7.21 E+6, and 1.44 E+6 particle/mL, with subsection (i) showing the aw Raman signal at the interested Raman shift, 1000 cm⁻¹, and with subsection (j) showing the Signal to Noise Ratio (SNR) for different sample concentrations, according to exemplary embodiments of the present disclosure.

FIG. 24 shows a schematic view of the neural network structure when, as shown in subsection (a), Raman, absorption, or concatenated Raman-absorption spectrum is used as the input, and as shown in subsection (b), both Raman and absorption spectra are used as separate units, with the number of layers shown for illustrative purposes and not reflective of actual values, according to exemplary embodiments of the present disclosure.

FIG. 25 shows a schematic view of the random forest model composed of multiple decision trees with either Raman, absorption, or concatenated Raman-absorption spectrum as the input, with “A” standing for the average, noting that the number of nodes and trees shown are for illustrative purposes and not reflective of actual values, according to exemplary embodiments of the present disclosure.

FIG. 26 shows raw and pre-processed Raman and absorption plots at two different concentrations, according to exemplary embodiments of the present disclosure.

FIG. 27 shows the comparison of the predictions of RF and CNN for one fold in the 5-fold cross-validation datasets when, as shown in subsection (a), the Raman spectrum, and as shown in subsection (b), the absorption spectrum, and as shown in subsections (c) and (d), the Raman-absorption absorption spectrum, are used as the input, with subsections (c) and (d) corresponding to a single and dual network, respectively, according to exemplary embodiments of the present disclosure.

FIG. 28 shows a comparison of the principal component analysis (PCA) plots at different concentrations for the Raman data (subsection (a)), the absorption data (subsection (b)), and the concatenated Raman-absorption data (subsection (c)), with subsection (d) showing a comparison of RF predictions with dimensionality reduction using PCA for different types of inputs (with R-A standing for the concatenated Raman-absorption), according to exemplary embodiments of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

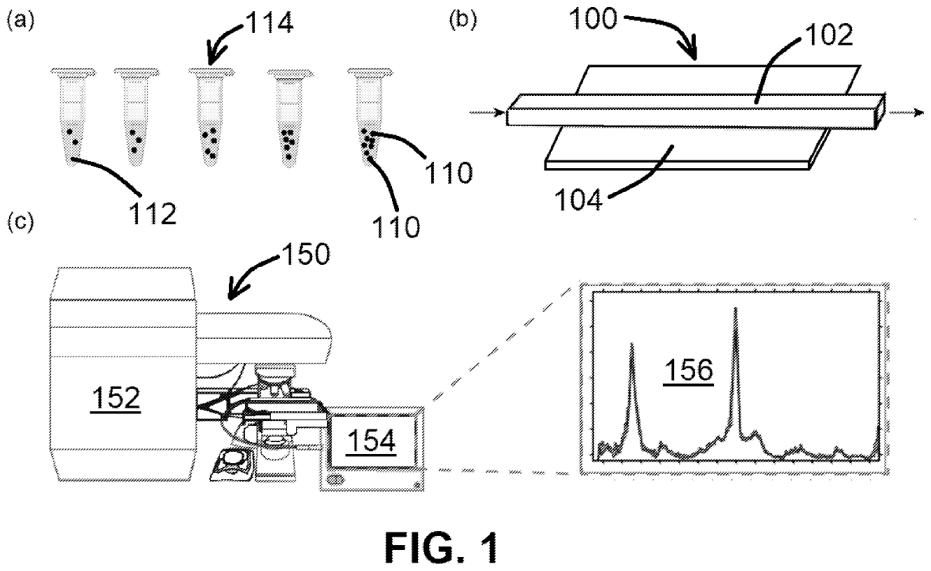

Raman spectroscopy enables non-destructive characterization of samples by providing detailed molecular fingerprints. Prior research included the development of a deep-learning method using Raman spectroscopy to detect bacteria, fungi, and mammalian cells in static dried-down conditions. Building on said earlier work, the present disclosure includes the use of Raman-based viral quantification in a continuous flow with a simple square quartz capillary tube setup (FIG. 1). Using this simple setup, viral loads were characterized at different flow rates.

As shown in subsection (b) of FIG. 1, exemplary devices 100 of the present disclosure comprise a capillary tube 102 affixed to a substrate 104, such as a glass slide, a LiNBO₃ wafer, or another substrate, as described in further detail herein. Capillary tubes 102 of the present disclosure have a square cross-section, as described in further detail herein, and can be affixed to substrates 104 using an epoxy, for example. An exemplary system 150 of the present disclosure can comprise an exemplary device 100 of the present disclosure along with one or more additional items, such as a Raman spectroscopy device 152, such as shown in subsection (c) of FIG. 1. A display 154 in communication with Raman spectroscopy device 152 is configured to display Raman spectroscopy data 156, such as shown in subsection (c) of FIG. 1. Said Raman spectroscopy data 156 can be indicative of microparticles or nanoparticles 110 suspended in a liquid 112, such as shown in the sample containers 114 in subsection (a) of FIG. 1.

The present disclosure demonstrates that Raman spectroscopy can characterize attenuated human cytomegalovirus (CMV) at a concentration between 4.5×10⁹ particles/mL to 2.9×10¹¹ particles/mL at flow rates between 50 μL/min to 250 μL/min within a square quartz. capillary tube 102. This method is versatile and can potentially be used for continuous monitoring of other VLPs in flow. As noted above, this is the first attempt to use a Raman spectroscopy-based tool to characterize virus at different concentrations in flow for viral vaccine manufacturing. This process has the potential to be expanded for continuous manufacturing of viral-based vaccine manufacturing as a process analytical tool.

### Viral Production and Purification

The vaccine virus was produced in human retinal pigment epithelium cells (ARPE-19) grown on Cytodex-1® microcarriers in a stirred-tank bioreactor using proprietary growth medium. After sufficient cell growth was achieved, the bioreactor was 80% medium exchanged, followed by viral inoculation. At fourteen days post-infection, the cell culture was harvested by removing the microcarrier beads through a strainer bag and subsequently clarified through Sartobind GF+1.2 μm filter. This clarified viral harvest was used for subsequent chromatographic studies.

During purification, single membrane runs for scouting experiments were performed with an Äkta Pure system (Cytica) using 10 mM histidine (addition of 150 mM NaCl, pH 7) equilibration buffer at 10 mL/min on 3 mL Sartobind Q nano membranes, for a total of 5 column volumes (CV). A wash step was performed for 15 CV at 180 mM NaCl and 80 mM Histidine, with pH of 6. For elution, a 15 CV gradient step to 1M NaCl (in 25 mM Histidine buffer, pH 7) was applied. In addition, alternative loads were performed using wash buffer as mix loading buffer. All collected fractions were analyzed off-line by SDS-PAGE and Apogee to estimate yields. For concentration, tangential flow filtration was performed using a 750 kDa hollow fiber (Cytiva, Upsala).

### Raman Analysis on Static Viral Fluids

To initially characterize the performance of the Raman microscope with liquid samples, liquid attenuated CMV was tested to obtain Raman spectra at different conditions (laser power and acquisition time). 20 μL of the attenuated CMV were placed on the stainless-steel substrate, as described in our previous work without drying. The stainless-steel substrate with the viral samples was analyzed immediately using the Renishaw inVia™ confocal Raman microscope (Renishaw plc, Wotton-under-Edge, UK). A 785-nm excitation laser with 0.5 mW power and 10 s acquisition time per measurement was used with 3 co-averages (i.e., 30 s total acquisition). To study the sensitivity of the Raman microscope with the liquid viral samples, different dilutions of viral samples (attenuated CMV in the concentration range of 4.5×10⁹ particles/mL to 2.9×10¹¹ particles/mL) were tested in the same setup. The supernatant of the buffer was obtained by centrifuging the attenuated CMV samples at 13,000 RPM for 15 mins using Thermo Scientific Pico™ 17 microcentrifuge (catalog #75002411) with MicroClick 24×2 fixed-angle microtube rotor (catalog #75005715) and used as a background control.

### Fabrication of Capillary Tube Setup for Viral Samples Under Flow

A single capillary tube 102 device 100 was fabricated to characterize the ability of the Raman microscope to detect viral particles in a continuous flow setup. We used a customized crystal-clear fused quartz square capillary tube 102 (Friedrich & Dimmock, Inc, NJ, USA) with the size of 2 mm×2 mm×50 mm (0.75 mm wall thickness). The square capillary tube 102 was bonded on a glass slide (substrate 104) using UV epoxy resin (60-7105, Epoxies Inc, RI, USA). Both ends of the quartz capillary tube 102 were connected to plastic tubes 130, 132 (ID 2 mm, 5233k112, McMaster-Carr, IL, USA), and the ends were sealed with the UV epoxy resin. The viral samples were pumped into the capillary tube 102 using a syringe pump 134 (70-3007, Harvard apparatus, MA, USA).

### Raman Analysis On a Square Capillary Tube With Flow

The capillary tube 102 setup referenced herein was used along with the syringe pump to mimic an on-line flow setup. The attenuated CMV with 2.9×10¹¹ was initially tested with the flow rate from 50 μl/min to 250 μl/min (0.2-1 mm/s). This flow rate was decided based on the viral vaccine production platform (320 cm/h˜0.9 mm/s). A syringe pump was used to provide the different flow rates inside the capillary tube 102. The overall capillary tube 102 setup was placed on the Renishaw inVia™ confocal Raman microscope mechanical stage. A 785-nm excitation laser with 0.5 mW power and 10 s acquisition time per measurement was used with 3 co-averages (i.e., 30s total acquisition) to obtain Raman spectra for the viral samples in the flow setup. To correct the baseline of Raman spectra, a baseline correction algorithm using Origin (OriginLab Corporation, Northampton, MA, US) software was implemented. The spectrum was smoothed using the adjacent-averaging method (10 points). Then, the spectrum was corrected by selecting a user-defined baseline mode with 20 points to find the peaks with spline interpolation method. The sensitivity of the Raman in a flow setup was tested by diluting the viral samples (attenuated cytomegalovirus CMV at the concentration of 2.9×10¹¹ particles/mL to 4.5×10⁹ particles/mL) with the same condition as mentioned above.

### Dynamic Light Scattering Analysis to Check the Effect of Laser Exposure

The effect of Raman laser exposure (785-nm excitation laser with 0.5 mW power with 30 s total acquisition time) on the viral particles was characterized using the dynamic light scattering (DLS) technique. This analysis will evaluate the effect of laser exposure on the size of the viral particles. The CMV samples at the highest concentration of 2.9×10¹¹ particles/mL and lowest concentration of 4.5×10⁹ particles/mL were used to evaluate this effect. 50 μl of viral samples were used for the laser exposure. Before and after the laser exposure, the size of the viral particles was analyzed using a disposable plastic micro cuvette (ZEN0040, Malvern MA, USA) in the Nano-S Zetasizer (Malvern, MA, USA). 500-nm fluorescent nanoparticles (CD Bioparticles catalog no. DNG-L023, USA) were used as a reference for the study.

### SDS PAGE and Western Blot Analysis After Laser Exposure

The functional impact of Raman laser exposure towards the viral protein samples was evaluated. Sodium dodecyl sulfate polyacrylamide gel electrophoresis (SDS-PAGE) and Western blotting was performed to analyze any changes that occurred during the laser exposure to the protein samples during Raman spectra acquisition. SDS-PAGE was performed using Invitrogen 14% Tris-glycine gel. Before and after the laser exposure, the samples were collected and were reduced in sample buffer containing DTT and heated at 70° C. for 10 min. The two CMV (before and after laser exposure) samples were loaded into gels with equal volume (10 μl). Electrophoresis was performed with constant voltage at 125 V for 1 hour and 55 mins. The gel was fixed with 12% trichloroacetic acid, washed with water, stained overnight with Gel Code Blue Stain, de-stained in water for >6 hours. The gel was scanned by a personal densitometer SI (model 375, GE Healthcare, USA).

To study the functionality of the proteins before and after the laser exposure, a non-reducing Western blot was performed using the SimpleWestern system (ProteinSimple, San Jose, CA). The technology was previously described in detail. Briefly, the two samples were diluted 4.3× in a sample buffer containing SDS and iodoacetamide and heated at 70° C. for 10 min. Then, automated SDS PAGE gel and Western blot were run. The critical CMV antigens, pentameric (gH-gL-UL128) and trimeric (gH-gL-gO) proteins, were probed using specific anti-gH, and chemiluminescence signal was collected.

### Static Liquid Testing Revealed C-H Stretching Peaks (2900 cm⁻¹) Change With the Concentration of CMV

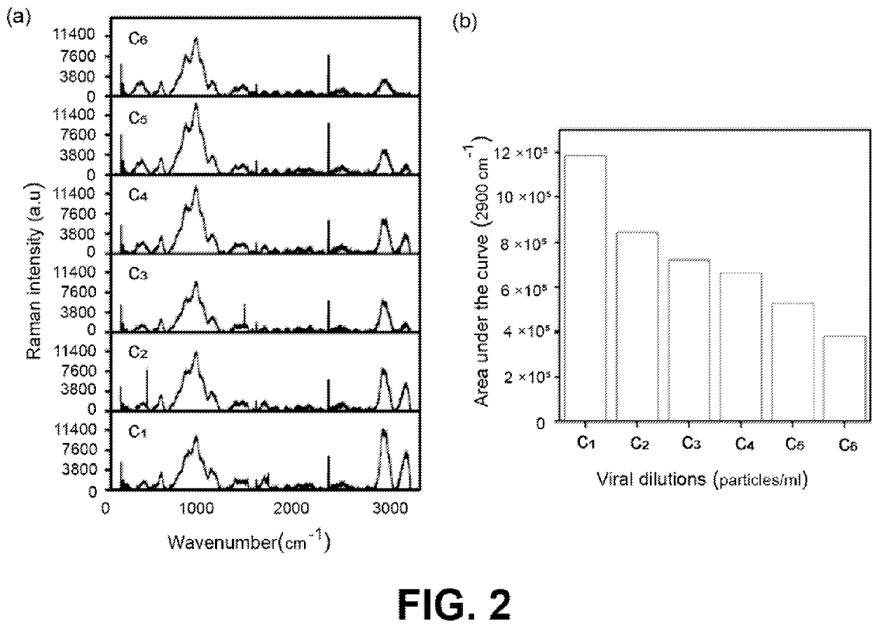

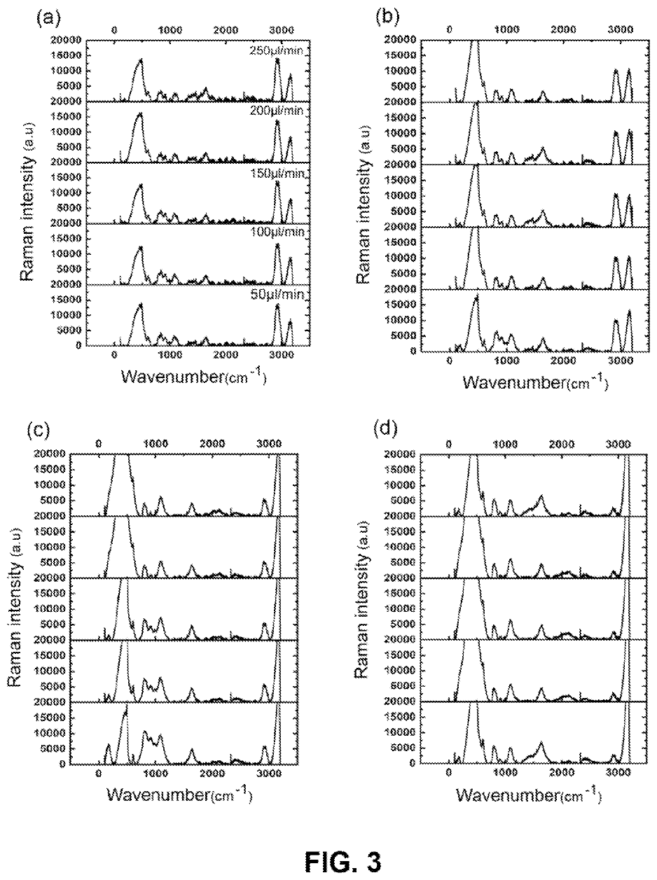

Initially, to study the possibility of employing Raman spectroscopy to characterize viral particles, the viral particles (8.96×10⁹ particles/mL to 2.9×10¹¹ particles/mL) were tested in a static condition using our previously reported method. The Raman spectra were obtained to find the Raman peak of interest for the CMV particles. The peak around 2900 cm⁻¹ was seen to be decreasing across different dilutions of the viral particles (see FIG. 2). This peak around 2900 cm⁻¹ is generally an important feature that is often neglected in viral particle detection using Raman spectroscopy; it corresponds to σ(C-H) stretching. Based on these results, it was identified that the peak around 2900 cm⁻¹ is the peak of interest to find the approximate concentration of the viral particles (see FIG. 3). This is the first report on developing a Raman spectra-based characterization of CMV particles. This observation can provide a significant advancement in direct virus monitoring if this platform is incorporated to continuously monitor viral particles. Thus, the ability of Raman spectroscopy to characterize samples under flow rates typically used in vaccine production was tested. With increasing concentration of the viral samples, it was observed that the Raman intensity increases linearly around the peak of 2900 cm⁻¹ as well as 1650 cm⁻¹ (Amide I), and 1350 cm⁻¹ (Amide III). Turbidity is not a crucial factor in the present study. The absorbance measurement of the samples was incorporated to correct the Raman signal for the effect of turbidity of the samples, and the results show no significant change in Raman signals.

### Flow Rate vs. Concentration of Viral Particles

A capillary tube 102 was used to test the ability of Raman spectroscopy to characterize virus particles in flow. COMSOL Multiphysics was used to calculate the flow velocity and shear stress within the capillary tube 102. Based on the numerical results, the Stokes number (ratio of a particle response time to a characteristic time of the flow in capillary) is very low for viral particles (Stk˜10⁻⁹), meaning that the viral particles follow the fluid streamlines and are transferred by bulk flow. So, CMV viral particles are uniformly distributed inside the capillary tube 102. Also, the range of shear stress inside the capillary tube 102 is 10⁻⁴ to 10⁻³ (Pa), which is lower than typical shear flow experiments involving human CMV samples and does not cause damage to the samples.

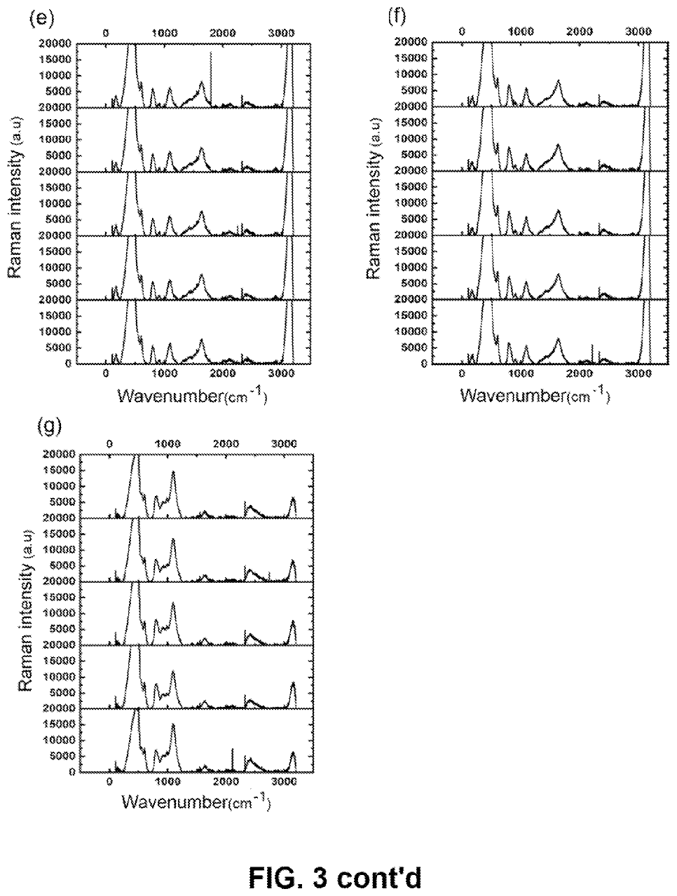

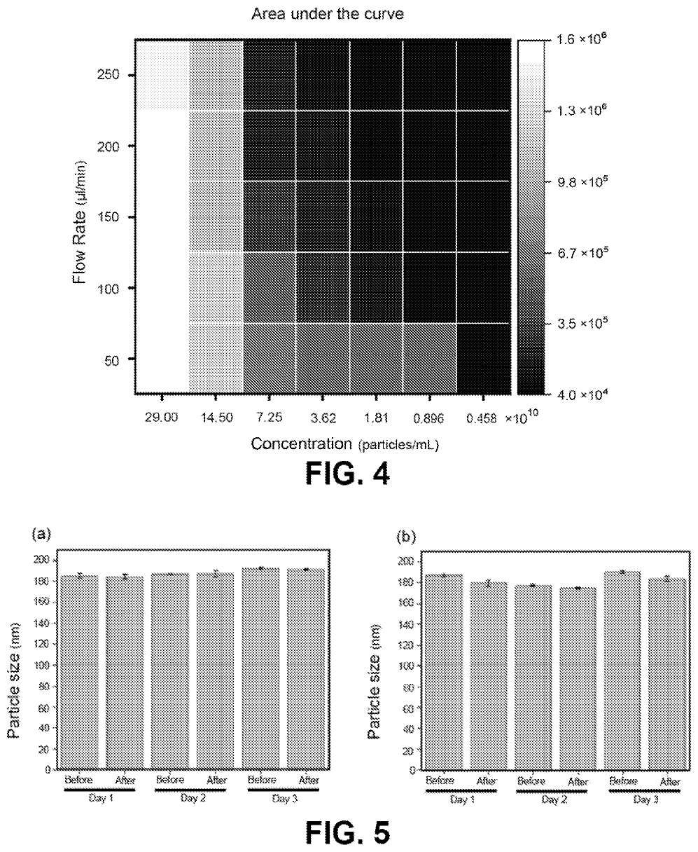

The CMV samples were loaded into the capillary tube 102 using the syringe pump with different flow rates from 50 μl/min to 250 μl/min using samples at different concentrations (4.5×10⁹ particles/mL to 2.9×10¹¹ particles/mL). The Raman spectra collected under all flow rates over all concentrations are depicted in FIG. 4. In all the concentrations, a Raman peak can be observed around 2900 cm⁻¹. Even in the higher flow rate, e.g., 1 mL/min, there is no change in the Raman peak around 2900 cm⁻¹. When changing the concentration (4.5×10⁹ particles/mL to 2.9×10¹¹ particles/mL), the last two dilutions show significantly less signal in the peak around 2900 cm⁻¹. The area under the curve of 2900 cm⁻¹ with different flow rates was depicted in a heat map for easy interpretation (see FIG. 5). This observation shows that Raman spectroscopy is functional at high flow rates (up to 1 ml/min) as long as the concentration of the viruses is high enough to be detected. Again, this is the first report to show the effect of Raman spectroscopy in CMV particle monitoring in a flow setup.

### Raman Spectroscopy is Non-Destructive

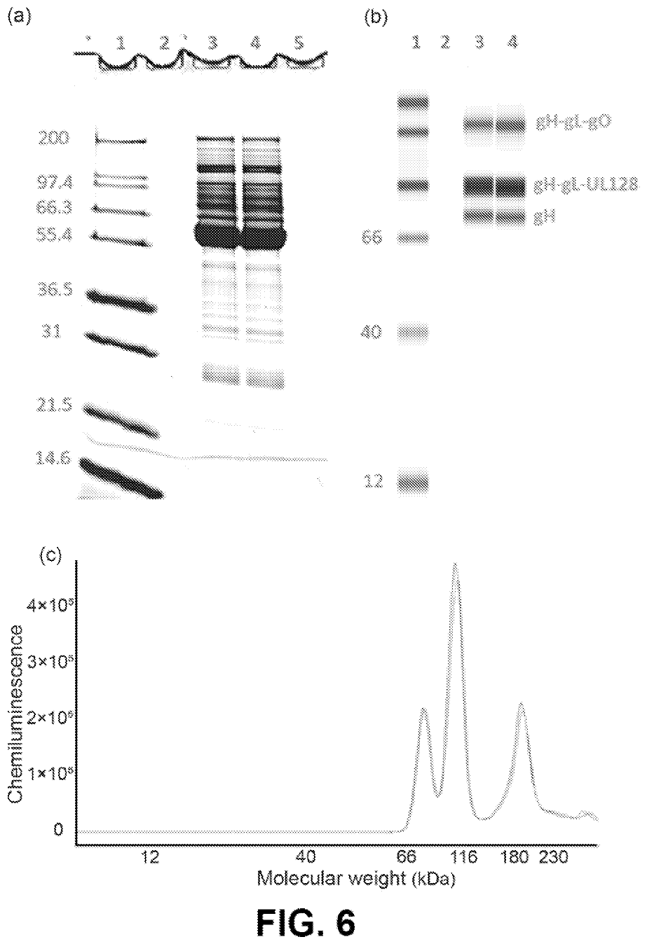

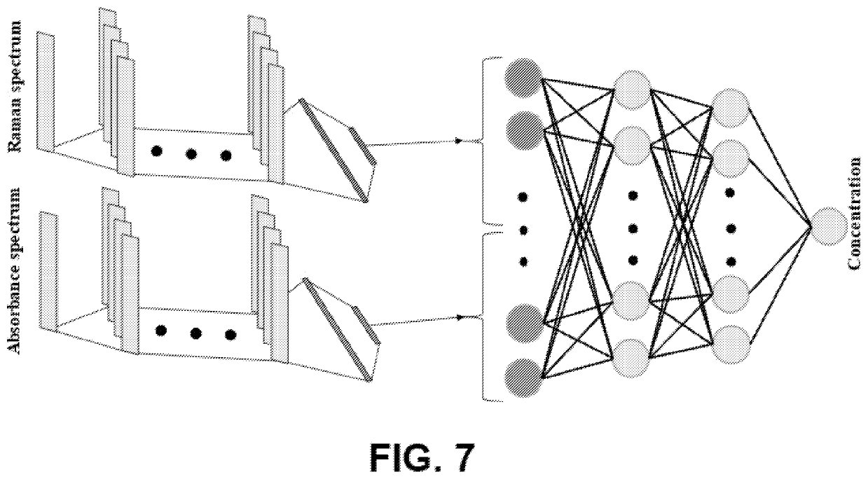

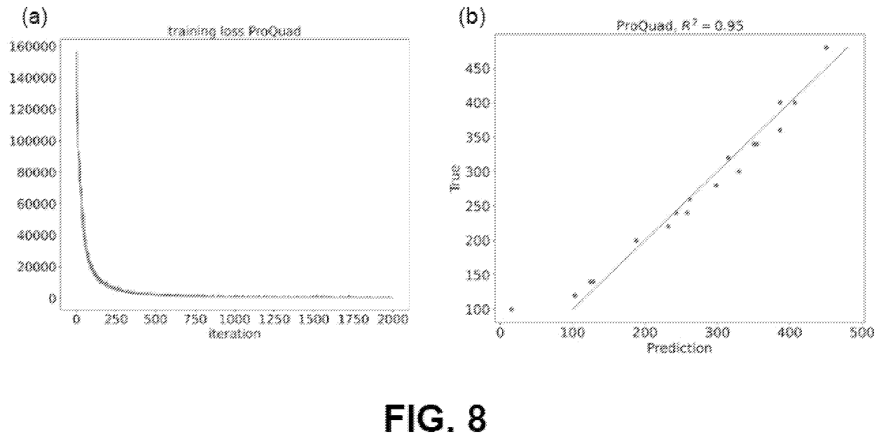

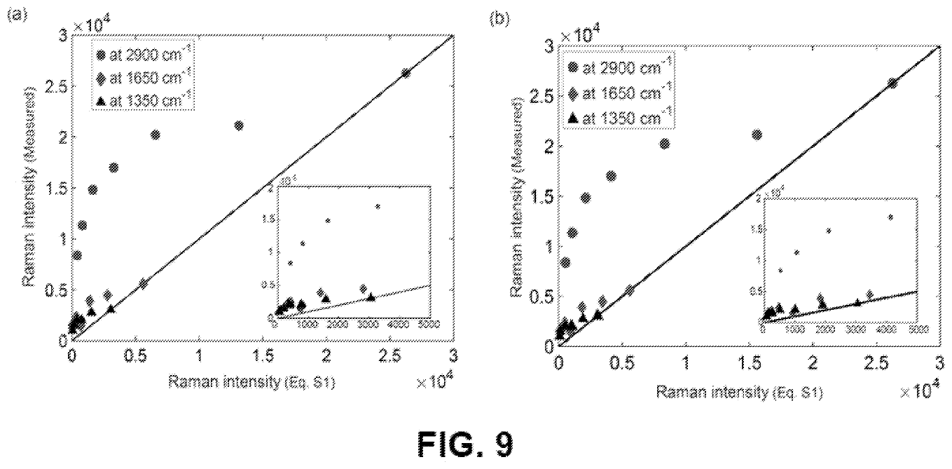

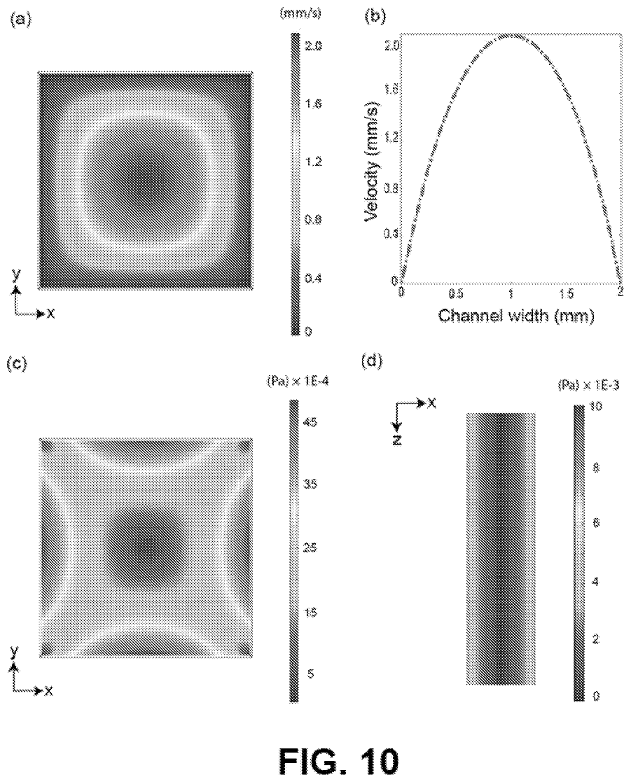

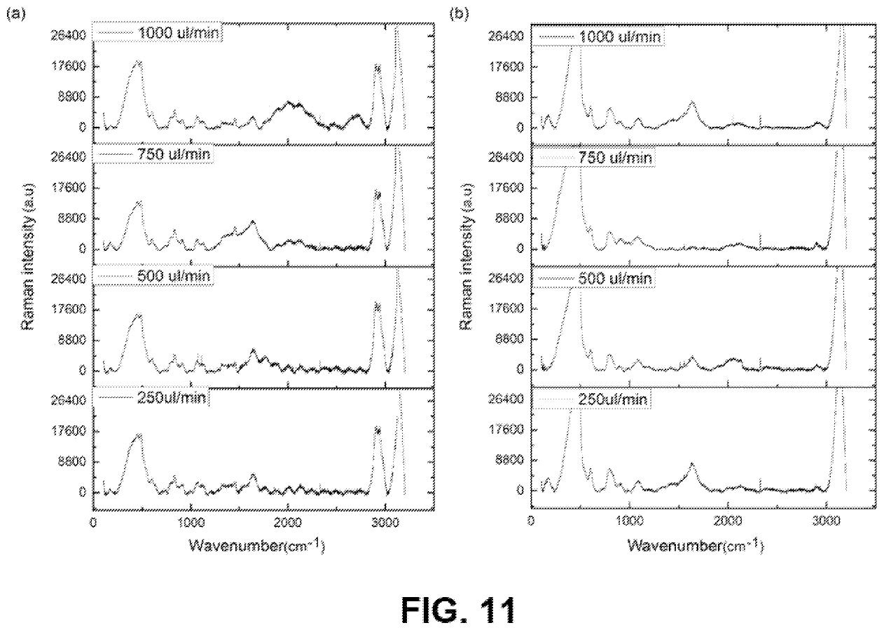

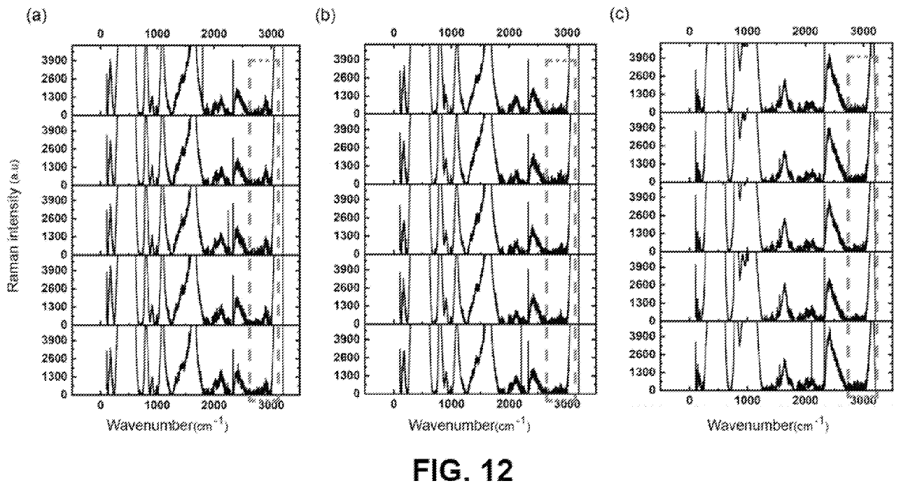

An important factor in developing a PAT tool is to ensure the tool is not destructive to the product (VLP/vaccine). Here the viral particles were exposed a laser with the excitation wavelength of 785 nm at 0.5 mW power. To ensure that the laser did not damage the CMV samples, the VLPs were analyzed before and after the laser exposure using SDS PAGE and Western blot. The effect of laser exposure on particle size using DLS was also determined. Based on the result from DLS, it was found that there are no significant changes in the size of viral particles before and after laser exposure (see FIG. 6). The Western blot and SDS-PAGE gel data of the viral particles before and after the laser exposure also show no significant changes in the protein quality (see FIG. 7). More importantly, the critical glycoprotein complexes (gH-gL-UL128 and gH-gL-gO) do not degrade before and after laser exposure, as illustrated by capillary western (see subsections (b) and (c) of FIG. 7). This is an important result that increases the possibility of adapting this technology as an analytical tool for continuous monitoring of VLPs for vaccine production.

The present disclosure demonstrates that Raman spectroscopy is a promising process analytical tool in the future of continuous manufacturing of biologics. The main bottleneck in continuous manufacturing is the lack of efficient PAT tools to satisfy the needs of analytical monitoring. Here, the Raman peak around 2900 cm⁻¹ was used as a marker for tracking attenuated CMV. This peak corresponds to the hydrogenic stretching of and aliphatic and aromatic CH groups. Other minor Raman peaks which are specific to particular viral particles around 500 to 1600 cm⁻¹ can also be taken into consideration. The incorporation of artificial intelligence and machine learning approaches along with the specific Raman peaks can provide more detailed information with the analysis of complex viral particles in the future.

The present disclosure also includes disclosure of the development of a Raman spectroscopy-based tool to characterize viral particles. Three different acoustic devices have been tested for the enrichment of nanoparticles. Also, Raman signals for different vaccine samples and attenuated human cytomegalovirus (CMV) at different concentrations and flow rates have been measured. Moreover, a machine learning package has been developed to characterize biological samples.

As referenced above, different samples with a variety of concentrations and flow rates were studied. The enrichment of nanoparticles was established through three different acoustic devices. Raman signals and machine learning algorithms have been obtained/developed to quantify biological samples. The general schematic of the bench setup is shown in FIG. 1 and as described in further detail above.

### Acousto-Capillary Device

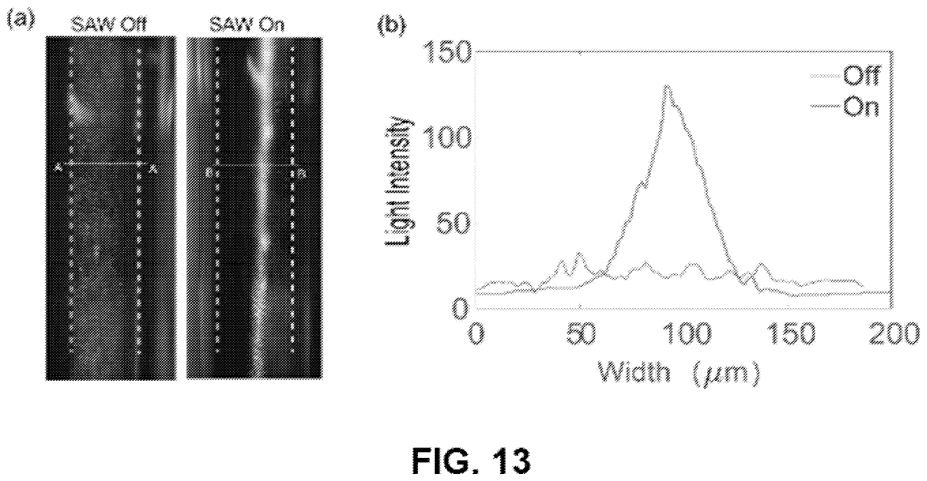

The acousto-capillary device works a half wavelength resonator and incorporates the acoustic radiation force and torsional force to create a single vortex mechanism for concentration of 500 (nm), such as shown in FIG. 13. FIG. 13 shows nanoparticles in capillary tube 102 with acoustic device off and on (subsection (a)), and the light intensity in channel cross sections increases (subsection b), whereby the intensity for device on is 129 and off is 20.6±2.95 (increased by factor: 129/20.6˜6.2), according to exemplary embodiments of the present disclosure.

### Acousto-Rotationary Device

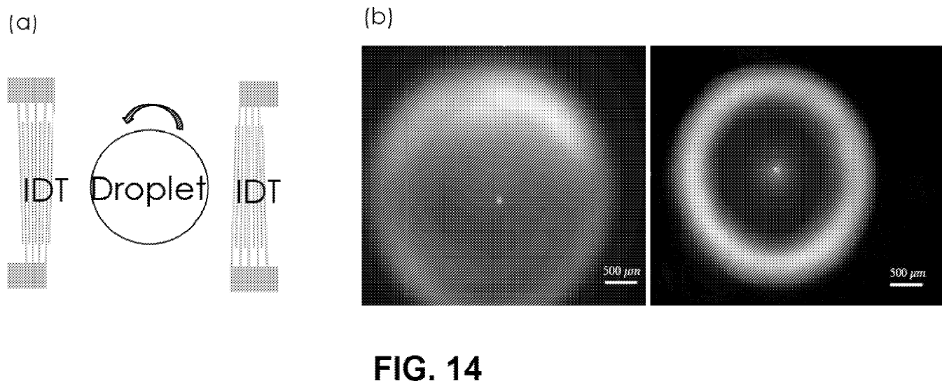

The ability of an acoustic device, equipped by slanted IDTs, was tested to concentrate particles. Different droplets have been tested (10-25 μL) to show the ability of particle concentration. As the acoustic force is applied, the droplet reaches a stable spin mode with a periodic rotational boundary deformation, which forms a “rotational capillary wave” propagating along the free surface of the droplet. In this mode, particles within the droplet migrate toward the center of the droplet following a dual-axis rotational trajectory, as shown in FIG. 14. FIG. 14 shows a schematic of droplet spinning between two IDTs (subsection (a)), and that the light intensity in droplets increases and 1 μm and 500 nm particles get enriched at the center of droplets (subsection (b)), with f=23.8 MHz, volume=13 & 10 μl, according to exemplary embodiments of the present disclosure.

### Acousto-Electronic Device

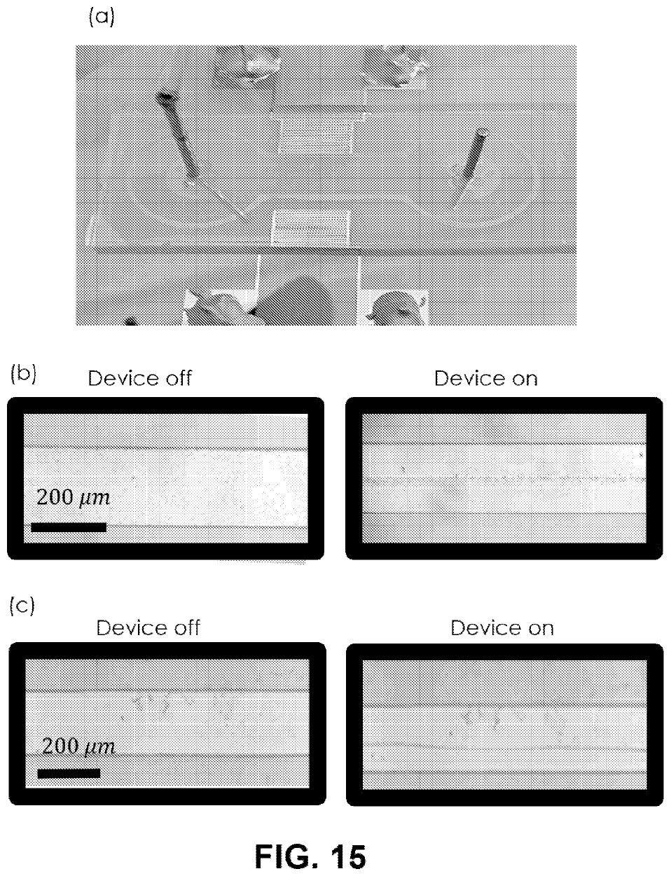

This device incorporates the effect of electrostatics and thermoviscous acoustics to create an electric and acoustic field in the liquid and piezoelectric substrate. Several forces such as drag, dielectrophoresis (a force is exerted on a dielectric particle when it is subjected to a non-uniform electric field), and acoustic forces are affecting the motion of nanoparticles inside the device. With a frequency of, f=14.352 MHz, a voltage of 36.4, and the polystyrene nanoparticle with a size of 1000 and 500 nm. FIG. 15 shows in subsection (a) a PDMS channel bonded on top of the device between to IDTs, in subsection (b) particle enrichment for 1 μm, and in subsection (c) 500 nm particles at the center middle of the channel, with f=14.353 MHZ, according to exemplary embodiments of the present disclosure.

### Raman signals for CMV Samples

As noted above, a Raman spectroscopy-based tool was used to quantify attenuated human cytomegalovirus (CMV) at a concentration between 4.5×10⁹ particles/mL to 2.9×10¹¹ particles/mL at flow rates between 50 μL/min to 250 μL/min within a square quartz capillary tube 102, such as shown in FIG. 2.

### Raman Signals for Vaccines

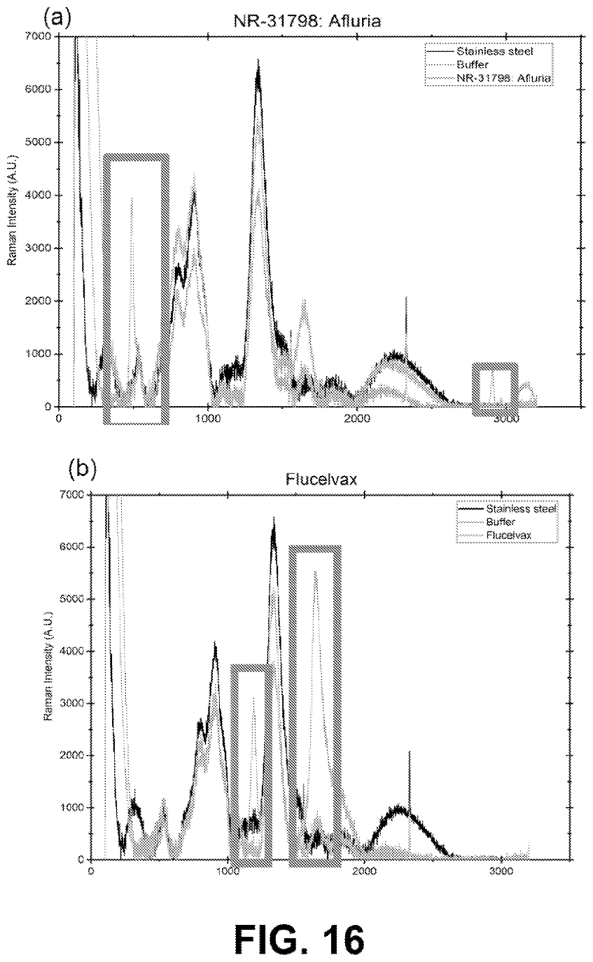

Raman signals were also collected for four different vaccines and their buffers. FIG. 16 shows Raman spectra for four different vaccines and their buffers, namely NR-31798 Alfuria (subsection (a)), Flucelvax (subsection (b)), NR-51702 Fluzone (subsection (c)), and IMOVAX® RABIES (subsection (d)), according to exemplary embodiments of the present disclosure.

### Raman Signals and Absorptions with Machine Learning

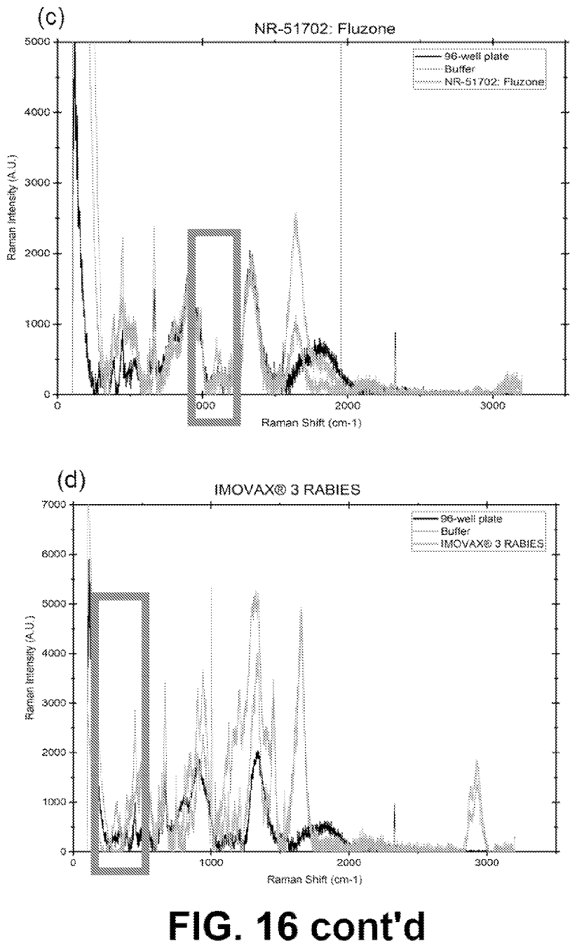

The incorporation of machine learning approaches along with the specific Raman peaks and absorption measurements provide more detailed information with the analysis of complex viral particles. FIG. 7 shows a double convolutional neural network (CNN) that uses Raman and turbidity (absorbance) spectrums as the inputs and predicts the concentration of the samples as the output, according to an exemplary embodiment of the present disclosure. FIG. 17 shows data comparing the developed algorithms versus obtained values for different viral concentrations, with absorption data shown at the top and Raman data shown at the bottom, according to exemplary embodiments of the present disclosure.

### Microparticle Manipulation

Manipulating micro and nanoparticles is essential in various fields, including biomedical research and environmental science. Particles such as polystyrene, gold, silver, and silicon, as well as nano bioparticles like extracellular vesicles are widely used in biomedical applications. This technology is useful in drug delivery, tissue engineering, biomaterials, and medical diagnostics. Researchers are constantly exploring new methods to manipulate these particles with accuracy and control. Various techniques such as dielectrophoresis, electrophoresis, inertial separation, pinched flow fractionation, magnetophoresis, and optical methods are commonly used to manipulate micro and nanoparticles. Each method has specific advantages and disadvantages, and they are often used in combination to improve their effectiveness.

Out of the aforementioned techniques, acoustics-based particle manipulation stands out for its high-throughput, noncontact, biocompatibility, simplicity, and cost-effectiveness. Free flow acoustophoresis and standing surface acoustic waves (SSAW)-based microfluidics have particularly gained popularity. These methods have been effectively applied for handling and processing diverse biosamples such as bacteria, cells, and viral particles. Moreover, acoustic streaming is commonly combined with radiation forces for manipulating microparticles or nanoparticles.

Two-dimensional acoustofluidics is a noteworthy acoustofluidic technology that enhances the acoustic radiation force in a straightforward yet effective manner. Antfolk et al. studied the use of two-dimensional bulk acoustic waves in a square channel, which allowed for the two-dimensional focusing of E. coli and polystyrene particles as small as 0.5 μm. Similarly, Mao et al. demonstrated the enrichment of nanoparticles using a square-shaped glass capillary tube with a surface acoustic wave (SAW) transducer. In another study, Masashi et al. showed that reducing the dimensions of the acoustic resonator improved efficiency, allowing successful manipulation of flowing 250 nm particles.

Although a 2D acoustic device in a larger channel has yet to be developed, the potential benefits of this technology are significant. Using a larger channel, the device can accommodate larger volumes, thus increasing its capacity for sample analysis. Additionally, a 2D acoustic device in a larger channel can reduce interference from the channel walls on sample measurements, leading to more accurate and reliable results. The present disclosure includes, a detailed explanation of the development of a 2D acoustic device on a larger scale and its application in Raman microscopy.

Optical microscopy, micro-particle image velocimetry (μPIV), laser-induced fluorescence (LIF), resonant light scattering (RLS), and electrical impedance sensing have been used to study particles, such as cells or nanoparticles, within microfluidic systems. These techniques can provide information about particles' size, concentration, morphology, and fluorescence properties within microfluidic systems. The choice of method depends on the specific research question and the properties of the particles being studied.

Raman spectroscopy, an optical method based on the inelastic scattering of light, known as the Raman effect, has become a widely used tool in numerous research fields, such as physics, material science, biology, and chemistry. Raman spectroscopy is an effective technique for the chemical characterization and identification of complex biological systems. This technique is non-invasive and ideal for working with fragile samples. It is suitable for analyzing highly concentrated solutions of (bio)molecules, bulk materials, as well as single cells and tissue samples.

The present disclosure demonstrates an optimized acoustofluidic device for Raman measurements for the first time. The device employs a 2D acoustophoretic particle focusing system, which uses acoustic forces to concentrate particles into a specific region for Raman analysis. To optimize Raman microscopy measurements of particles focused by the device, we addressed various challenges related to Raman spectroscopy of the capillary tube 102, which can act as an obstruction to measuring the Raman signal of the sample. Specifically, the capillary tube 102 dimensions and the microscopic magnification were optimized to minimize the background noise and maximize particle focusing efficiency. Our optimized acoustofluidic device for Raman measurements provides a new means of enhancing the sensitivity and accuracy of Raman analysis, with potential applications in a range of research fields, including biology, medicine, and materials science.

### Operating Principle

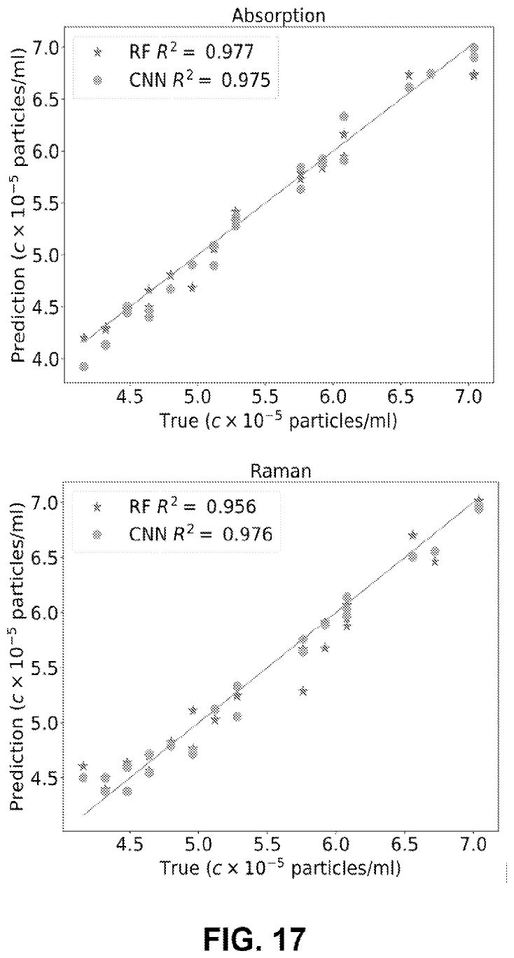

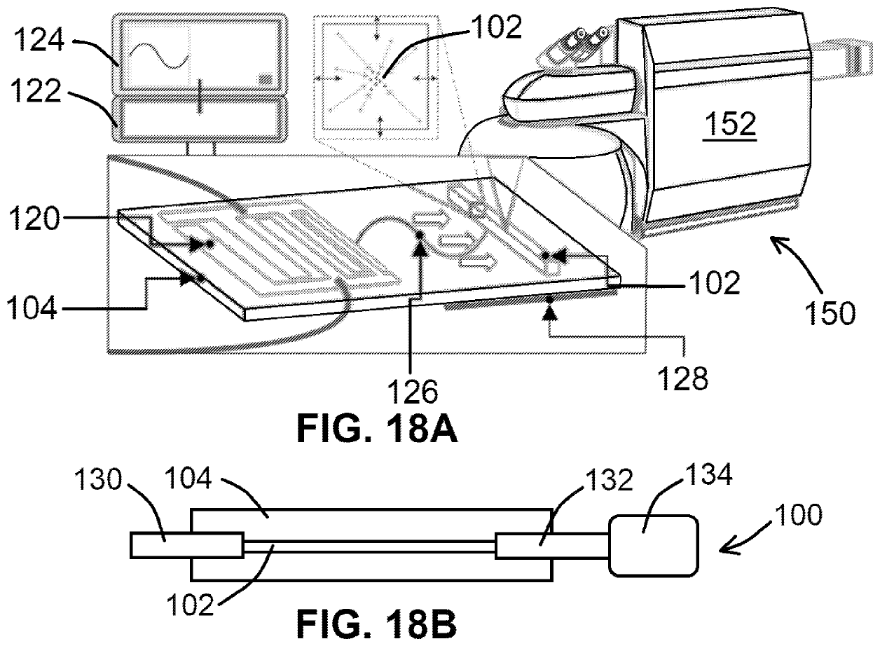

FIG. 18A shows a schematic of an acoustofluidic-based nanoparticle focusing device 100 and experimental setup (system 150), namely a three-dimensional schematic illustration of the device and Raman measurement. FIG. 18B shows details of an exemplary device 100 of the present disclosure. FIG. 19 shows an image of the acoustofluidic device 100.

This study examined a glass capillary tube 102 with a square cross-section placed on a Lithium Niobate (LiNBO₃) substrate 104, including an Interdigital Transducer (IDT) electrode 120, as shown in FIG. 18. The capillary tube 102 contains a suspension of particles (microparticles or nanoparticles 110 suspended in a liquid 112, such as shown in subsection (a) of FIG. 1), and the device 100 is activated by a radio signal from a wave function generator 124 and an amplifier 122 that is connected to the IDT 120 on the substrate 104. The SAW 126 produced by the IDT 120 travels through the LiNBO₃ substrate 104 and a thin layer of UV epoxy to reach the capillary tube 102. The SAW 126 causes the bottom of the capillary tube 102 to vibrate, which generates an acoustic force that acts on the fluid 112 and particles 110 inside. Consequently, the particles 110 in the acoustophoretic system 150 are propelled by the acoustic radiation force and viscous drag force from the acoustic streaming flow that is formed near the particles 110. A stainless-steel plate 128 was attached underneath to eliminate the interference of Raman signals from other materials beneath the wafer (substrate 104), allowing the Raman signal to contain only signals from stainless steel 128, epoxy, glass capillary 102, and the LiNBO₃ substrate.

FIG. 18B shows an exemplary embodiment of a device 100 of the present disclosure. As shown therein, device 100 comprises a capillary tube 102 affixed to a substrate 104 using epoxy, with a first plastic tube 130 coupled to one end of the capillary tube 102 (also using epoxy to seal), and with a second plastic tube 132 coupled to a second end of the capillary tube 102 (sealed with epoxy). A pump 134 can be used to pump liquid 112 having microparticles or nanoparticles 110 therein through the tubes 130, 132 and the capillary tube 102 so that the microparticles or nanoparticles 110 can be examined using Raman spectroscopy, such as shown in FIG. 18A.

FIG. 19 shows a top view of an exemplary IDT 120 of the present disclosure. As shown therein, IDT 120 comprises a first and a second vertical bar 140, 142, with alternating horizontal fingers 144 extending therefrom. Such an embodiment allows a SAW 126 to be produced by the IDT by way of the radio signal from an AC power supply 122 and a wave function generator 124, which causes the capillary to vibrate, permitting effective Raman spectroscopy on the microparticles or nanoparticles 110 within the capillary tube 102.

The streaming velocity varies with the wave's amplitude and applied frequency. The drag force from the acoustic streaming flow migrates the particle circumferentially as

$${F}_{drag} = 6 π a η ( v - {v}_{s} ) , \qquad ( 1 )$$

where a is the diameter of the particle, η is the viscosity, v is the velocity of the fluid, and vₛ is the particle velocity.

The gradient of an acoustic potential U_{rad} gives the acoustic radiation force, leading to the radial movement of particles

$${F}_{rad} = - ∇ {U}_{rad} , \qquad ( 2 )$$

$${U}_{rad} = \frac{2 π}{3} {a}^{3} ( \frac{〈 {p}^{2} 〉}{{ρ}_{0} {c}_{0}^{2}} {f}_{1} - \frac{3 {ρ}_{0} 〈 {v}^{2} 〉}{2} {f}_{2} ) , \qquad ( 3 )$$

$$where ,$$

$${f}_{1} = 1 - \frac{{ρ}_{0} {c}_{0}^{2}}{{ρ}_{s} {c}_{s}^{2}} , {f}_{2} = 2 ( \frac{{ρ}_{s} - {ρ}_{0}}{2 {ρ}_{s} + {ρ}_{0}} ) . \qquad ( 4 )$$

Here, p is the pressure, angle brackets indicate time averages, ρ₀ is the density of the fluid, ρₛ is the density of particle, c₀ is the speed of sound in the fluid, cₛ is the speed of sound in the particle.

The acoustic radiation force results from the scattering of acoustic waves on particles, while the drag force is due to resistance to this motion. Based on the equilibrium of this driving force and drag force, the unidimensional concentration is confined to a relatively large size, coined for the critical particle diameter. The critical particle diameter determines the transition from radiation-force-dominated particle motion to acoustic streaming.

$$2 {a}_{c} = \sqrt{\frac{12 sv}{πΦ f}} , \qquad ( 5 )$$

where 2a_{c} is the critical particle diameter, s is a factor related to the channel geometry, v is the kinematic viscosity of the media, Φ is the acoustic contrast factor, and f is the frequency of the applied acoustic field. Antfolk et al. stated that, at a frequency of 3.19 MHz, the resolution limit in their experimental setup using polystyrene particles was approximately 1.6 μm.

Two-dimensional radiation can help improve the limitation of detection of particles and enable faster accumulation of particles. This is because, in a two-dimensional radiation field, particles can be manipulated using both the radiation force from the bottom wall and the radiation force from the sidewalls. A square-cut capillary tube 102 can generate 2-dimensional radiation due to a torsional vibration of the capillary tube 102. A torsional vibration is induced in the bottom wall of the square-cut capillary tube 102, which is then transmitted through the capillary tube 102 to the side walls. The side walls also vibrate, but with a π/2 phase shift relative to the bottom wall vibration, resulting in a two-dimensional radiation field that can be used to manipulate particles and cells in the microchannel. The successful generation of the two-dimensional radiation field using this technique depends on various factors such as the capillary tube 102 size, the capillary tube 102 walls' thickness, and the capillary tube 102 material's properties, including its moment of inertia. The optimal dimensions and properties can be determined experimentally based on the application and the size and type of particles or cells being manipulated.

Raman spectroscopy is an analytical technique using inelastic light scattering to identify molecules' vibrations in a sample. However, Raman spectroscopy is a low-sensitivity technique, which means the required concentration of the molecule of interest is relatively high. Also, the signal can be easily overpowered by fluorescence or other background noise. To solve these problems, we need to increase the concentration of the samples.

Acoustic enrichment is a promising technique for increasing the concentration of molecules and improving the detection limit, allowing measurements of low concentration samples. In this regard, selecting a smaller capillary tube 102 can aid in detecting molecules more effectively. However, the glass capillary tube 102 can produce a Raman signal that can interfere with particle detection. The distance between the focal point and the capillary tube 102 wall can affect signal quality, suggesting that larger inner diameters are more advantageous than smaller ones. These two factors can be contradictory, and it is crucial to find the optimal capillary tube 102 dimensions that strike a balance between signal reduction and effective particle detection.

The magnification of a Raman microscope significantly impacts the signal-to-noise ratio and the quality of measurement in terms of focal depth. Increasing the magnification increases the spatial resolution, which improves the ability to detect small particles. However, higher magnification reduces the amount of light collected, decreasing the signal-to-noise ratio. Additionally, the quality of measurement in terms of focal depth is also affected by the magnification. Higher magnification can result in a smaller focal depth, making it easy to analyze samples in the capillary tube 102 by blowing up the background Raman signal. Therefore, choosing an appropriate magnification that balances spatial resolution, signal-to-noise ratio, and focal depth is essential.

### Device Fabrication

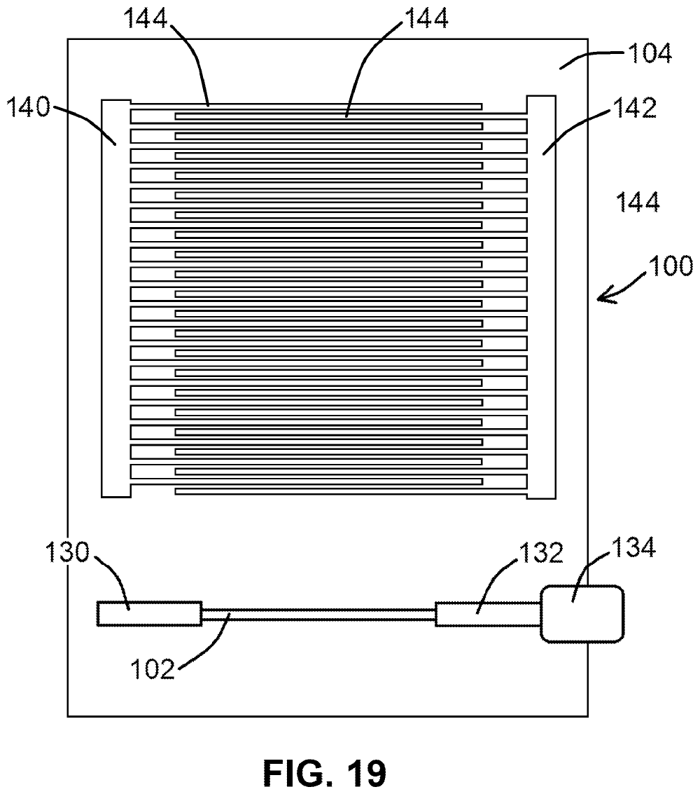

FIG. 20 shows a chronologically ordered schematic illustration of the fabrication steps, whereby subsection (a) shows that the process uses a lithium niobate (LiNBO₃) wafer (substrate 104), subsection (b) shows the spin-coated photoresist onto the LiNBO₃ wafer, subsection (c) shows the maskless photolithography for the patterning, subsection (d) shows developing the photoresist, subsection (e) shows metal deposition, subsection (f) shows lifting the photoresist off, subsection (g) shows the application of epoxies (whereby conducting epoxy is applied to the electrode, while clear UV epoxy attaches the glass capillary tube 102 to the wafer (substrate 104), and subsection (h) shows the conclusion of fabrication with the bonding of the capillary tube 102 to the wafer (substrate 104).

The devices used in this study were fabricated by bonding a square cross-section glass capillary tube 102 to a 4-inch 128° Y-cut lithium niobate wafer embedded with an interdigital transducer (IDT). The wafer was 500 μm thick, and the IDT was fixed using electron beam evaporation of two metal layers (titanium (Ti) and gold (Au)), with thicknesses of 10 nm and 100 nm, respectively, after conventional photolithography. The glass capillary tubes 102 used in the study had dimensions of 400 μm, 600 μm, and 800 μm, with varying wall thicknesses of 100 μm, 150 μm, and 200 μm, respectively. The IDT consists of two structures, each with 20 fingers. The width of fingers and the spacing between fingers ranged linearly, with a difference of 2 μm, providing operating frequencies ranging from 3.1 MHz to 4.6 MHz, from 2.2 MHz to 2.8 MHz, and from 1.7 MHz to 2.1 MHz for 400 μm, 600 μm, and 800 μm capillary tubes 102, respectively. The capillary tubes 102 were placed on the wafer 5 mm from the IDT, and UV epoxy (Crystal clear ultraviolet Curing epoxy resin, Limino, USA) was applied on the substrate using stencil tape to regulate the epoxy thickness. The epoxy was solidified by exposing it to UV light for 15 minutes.

### Determination of Raman Signal From the Particle and Background Raman Signals Via Raman Microscope

To determine the Raman spectra of polystyrene (PS) particles, dried 500 nm PS beads were tested to obtain their Raman spectra. The PS samples were placed on a stainless-steel substrate and analyzed using the Renishaw inVia™ confocal Raman microscope (Renishaw plc, Wotton-under-Edge, UK). In addition, the Raman spectra of Lithium Niobate substrate and UV epoxy was also measured with and without the glass capillary tube 102. For the measurements, we used a 785 nm excitation laser with a power of 30 mW and an acquisition time of 10 seconds. To evaluate the device's performance for Raman measurement, enriched particles with different concentrations were tested inside the capillary tube 102 channel with different dimensions and obtained Raman spectra.

### Experimental Setup

The experimental setup is depicted in FIG. 18. A multi-channel functional generator was used to generate the electrical signal for actuation with different frequencies, which was then amplified using a power amplifier. The amplified signal was set to less than 100 V and applied to both ends of the electrodes, causing vibration of the LiNBO₃ wafer (substrate 104). A stainless-steel plate was attached underneath to eliminate the interference of Raman signals from other materials beneath the wafer (substrate 104), allowing the Raman signal to contain only signals from stainless steel, epoxy, glass capillary tube 102, and LiNBO₃. Polystyrene nanoparticles were injected into the capillary tube 102 using a syringe (pump 134), diluted in 0.1% tween 20 solutions to various concentrations. The input laser power was 150 mW. Raman measurements were done for three different conditions/locations: (1) the device on in the concentrated region, (2) the device on in the non-concentrated region, and (3) the device off.

### Numerical Modeling

The enriching ability of the capillary was numerically studied using COMSOL Multiphysics by calculating the flow velocity and shear stress within the capillary tubes 102 with different dimensions.

### Determination of the Particle Line Width Via Microscope

Experiments were performed with capillary tubes 102 of varying dimensions to assess the rate at which particles are focused. The applied voltage was restricted to 100 V. 500 nm fluorescent polystyrene particles (Fluospheres™ carboxylate-modified microspheres, Invitrogen) diluted in DI water were employed. The solution concentration was 1.44 E+08 particles/mL. The images were captured using a Nikon microscope equipped with an sCMOS camera (Zyla 5.5). The laser power for fluorescent was fixed to allow us to infer that the concentration of beads is directly proportional to the measured fluorescent intensity.

### Results and Discussion

### Optimizing Focusing Performance Through Channel Width Variation

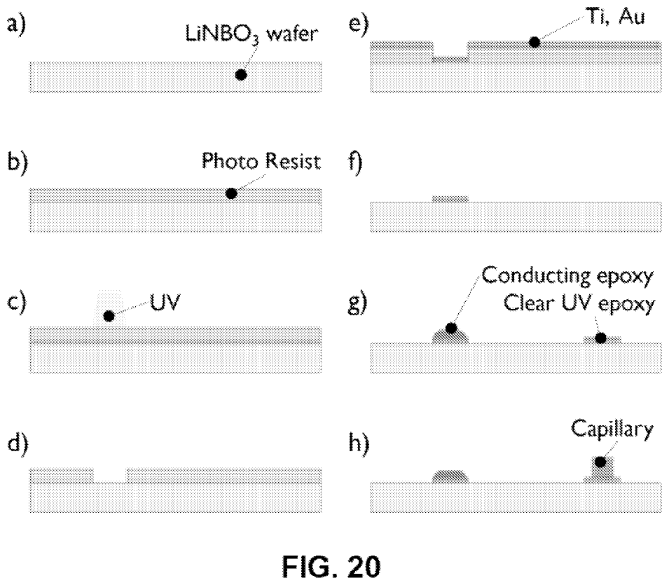

FIG. 21 shows time-lapse images and intensity of 500 nm fluorescent particles under the acoustic field, with subsection (a) and subsection (b) showing time-lapse images of the beads' motion in 400 μm and 800 μm capillary tubes 102, respectively (whereby the scale bar indicates 200 μm, and 400 μm, respectively), with subsection (c) showing the Gaussian fitting of the intensity at different times in the 800 μm capillary tube 102, and with subsection (d) showing the intensity of the peak concentration (i.e., Gaussian curve's maximum point) as a time function.

FIG. 21 demonstrates the process of concentrating 500 nm fluorescent particles in capillary tubes 102 of different sizes. Subsection (a) of FIG. 21 illustrates the time-dependent accumulation of particles in a 400 μm capillary tube 102, with the maximum fluorescent intensity reached at the capillary tube's 102 center after 80 seconds. Subsection (b) of FIG. 21 shows that it takes 450 seconds to concentrate the particles in the largest capillary tube 102, which is 800 μm in outer diameter. By calculating the average intensity at each position along the capillary tube 102 width and fitting it with a second-order Gaussian function, subsection (c) of FIG. 21 displays the gradual increase of the maximum intensity over time. Subsection (d) of FIG. 21 demonstrates the saturation of the peak concentration. One can define the accumulation time, which is 80 seconds for the 400 μm capillary tube 102 and 450 seconds for the 800 μm capillary tube 102.

The findings suggest that the capillary tube 102 size influences the effectiveness of the acoustic streaming and concentration process. Firstly, the fluid within the capillary tube 102 acts as a dampener, which limits the efficient transmission of vibrations from the bottom to the top of the capillary tube 102. Secondly, the particles must travel a greater distance in larger capillaries, which leads to longer concentration times. As predicted, the accumulation of particles in the largest capillary tube 102 (800 μm) took about five times longer than in the smallest capillary tube 102 (400 μm).

### Raman Measurement on Capillaries with Various Dimensions and Various Raman Microscopic Magnification

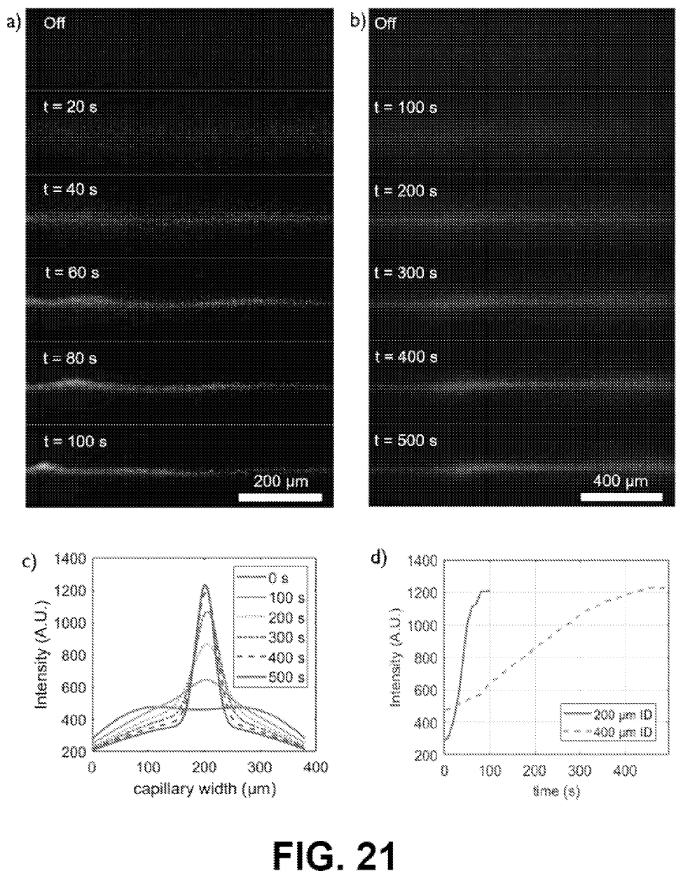

FIG. 22 shows the Raman signals obtained from capillary tubes 102 of different sizes and polystyrene beads, with subsection (a) and subsection (b) showing the Raman signals obtained from 800 μm and 400 μm capillary tubes 102, respectively, bonded on LiNBO₃ wafer (substrate 104), with subsection (c) showing a comparison of Raman intensity between capillary tubes 102 of various dimensions, with subsection (d) showing Raman intensity at different magnifications, and with subsection (e) showing the Raman intensity obtained from polystyrene beads and epoxy on LiNBO₃ was normalized by the area under the curve.

It is necessary to minimize the Raman signal from capillary tubes 102 to detect the Raman signal of particles of interest. Glass capillary tubes 102 have a consistent Raman intensity across the entire range of Raman shift measured, but the capillary tube 102 size affects the Raman signal. Subsections (a) and (b) of FIG. 22 show the Raman spectroscopy of a capillary tube 102 of 800 μm and 400 μm, respectively, both attached to LiNBO₃ with UV epoxy. The Raman shift range measured was 830 cm⁻¹ to 1900 cm⁻¹, and the x-axis represents the Raman shift while the y-axis represents the measurement position within the capillary tube 102. The capillary tube 102 wall had a higher Raman intensity than the center of the capillary tube 102. Since the particles of interest are focused on the center of the capillary tube 102, it is crucial to reduce the Raman intensity of the capillary tube 102 in the center to obtain accurate measurements. Subsection (c) of FIG. 22 shows the Raman spectroscopy of the center of the capillary tubes 102 of different sizes, visualized using an 8^{th}-order Gaussian fitting. The 400 μm capillary tube 102 had a higher intensity at both the capillary tube 102 wall and center compared to the 800 μm capillary tube 102, indicating that the 800 μm capillary tube 102 is better than the 400 μm capillary tube 102. These measurements were performed using a 300 mW laser power and a lens with 50× magnification.

Choosing the appropriate magnification is crucial for effectively capturing the Raman signal from a sample. Subsection (d) of FIG. 22 shows how magnification affects the reduction of the Raman spectroscopy of the capillary tubes 102, which can serve as background noise. By using high magnification, we demonstrated that the Raman spectroscopy of the capillary tube 102 could be minimized by reducing the focal depth.

The polystyrene particles utilized in our research exhibit Raman peaks at 1002, 1030, 1155, 1182, 1205, 1585, and 1603 cm⁻¹, as illustrated in subsection (e) of FIG. 22. The Raman spectroscopy of the polystyrene particles was obtained by drying the polystyrene solution on a stainless-steel plate and then normalizing the results based on the area under the curve. Additionally, a combination of the epoxy and LiNBO₃ wafers (substrates 104) display Raman peaks at 1001 and 1449 cm⁻¹. This implies that capillary tubes 102 with a larger inner diameter and thicker wall have an advantage in reducing the impact of the epoxy and wafer.

### Raman Spectroscopy of Dilute Particles Using the Device

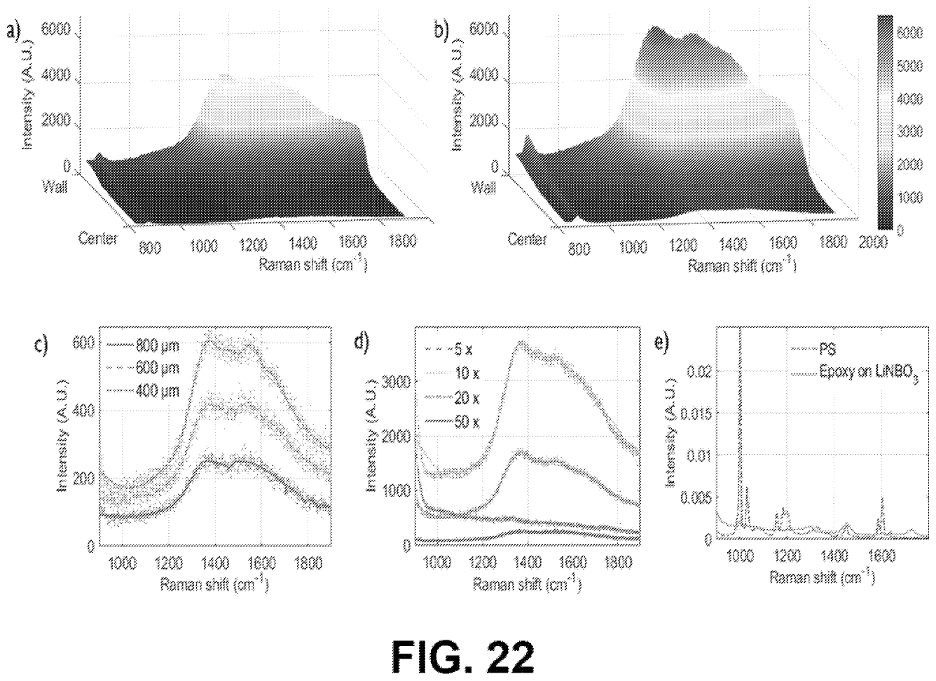

FIG. 23 shows Raman measurements on the focused region in solutions of various concentrations, with subsection (a) showing a normalized Raman measurement of the focused region of 1.44 E+10 particle/mL (whereby the solid line measures the concentrated region, while the dashed line corresponds to the device off), and subsections (b), (c), (d), (e), (f), (g), and (h) showing subtraction of the normalized Raman intensity when the device off from normalized Raman intensity from the focused region, where the initial input concentration was 1.44 E+10, 7.21 E+8. 1.44 E+8, 7.21 E+7, 1.44 E+7, 7.21 E+6, and 1.44 E+6 particle/mL, with subsection (i) showing the aw Raman signal at the interested Raman shift. 1000 cm⁻¹, and with subsection (j) showing the Signal to Noise Ratio (SNR) for different sample concentrations.

In the previous section, the Raman spectroscopy was measured with the device on and off, using a frequency of 1.846 MHz and a magnification of 50×, with an 800 μm capillary tube 102. Subsection (a) of FIG. 23 displays the Raman spectroscopy of polystyrene nanoparticles in a solution with a concentration of 1.44E+10 particle/mL, with the solid line representing the particle-focused region under the AC, and the dashed line representing the Raman spectroscopy with the device off. Each data consists of five independent measurements, and the shaded area indicates the standard deviation. The intensities were normalized based on the area under the curve. The significant peak from the particles, such as the 1000 cm⁻¹ peak, is visibly smaller when the device is off. Subsection (b) of FIG. 23 shows the difference between the device on and off, which is the Raman signal from the polystyrene particles. Similar to subsection (a) of FIG. 23, the shaded area indicates the standard deviation of the data. Subsections (c) through (h) of FIG. 23 exhibit the Raman signal of the polystyrene particles in the focused region. The peak around 1000 cm⁻¹ reduces as the concentration of polystyrene particles decreases.

The Gaussian fitting of the raw Raman intensities around the peak of interest at 1000 cm⁻¹ is displayed in subsection (i) of FIG. 23. For each concentration, five measurements were conducted, and the shaded areas represent the standard deviation of the results.

Subsection (j) of FIG. 23 shows the signal-to-noise ratio (SNR) of five measurements for each concentration. Without AC, marked as orange squares, the particles are uniformly distributed. The Raman measurement of the center of the capillary tube 102 when the device is off shows a negligible peak even for concentrations until as high as 1E+9. However, with an increase in concentration, the particles are captured by analyzing the Raman signal. When the concentration is 7.21E+8, the signal-to-noise ratio is 1.13. The signal-to-noise ratio was calculated by the peak height at 1.0×103 cm⁻¹ over the height of the background signal. The signal-to-noise ratio increases to 1.92 when the concentration is 1.4E+10. When the device is on at an appropriate frequency, 1.846 MHz, the detection limit improves to concentrations as low as 1.44E+6 particle/mL, which has an SNR of 1.17. The SNR increases with increasing initial concentrations of the solution. SNR were 1.23, 1.44, 2.79, 5.62, 5.39, 8.16 for the concentration of 7.21E+6, 1.44E+7, 7.21E+7, 1.44E+8, 7.21E+8, and 1.44E+10, respectively. The error bar indicates the standard deviation of measurements. Since the particles are not uniformly distributed along the capillary tube 102 when the device is on, some fluctuations in the Raman signal are observed. The Raman signal from the initial concentration of 1.44 E+8 in the concentrated region of particles showed an excellent result.

### Discussion

As noted above, the present disclosure includes disclosure of an integrated approach of nanoparticle manipulation based on 2D acoustofluidics and Raman spectroscopy to improve the detection limit. Experimentation proved that by increasing the capillary tube 102 size, optical clearance of the background Raman signal from the capillary tube 102 improves the Raman spectroscopy measurements despite the reduction in acoustic radiation force. The disclosed integrated approach can distinguish solutions with a concentration of 1.44E+6 particle/mL for 500 nm polystyrene nanoparticles through Raman measurement. In contrast, without the device 100, the particles are only detectable from a concentration of 7.21E+8, which is 500 times greater. The ability to manipulate sub-micrometer particles and inline measurements under a Raman microscope opens up new applications in various fields.

### Additional Data Acquisition

All the samples prepared in this section are based on the ProQuad®, which is a sterile, lyophilized, preservative-free, live virus vaccine that contains measles, mumps, rubella, and varicella-zoster viruses. ProQuad® (manufactured by Merck & Co., Inc., West Point, PA) was procured from the Purdue College of pharmacy and stored at −20° C. Linear dilutions of the ProQuad® vaccine were prepared with a step size of 4% and an initial concentration of 7.20E+05 plaque forming units/ml (PFU/mL) (Lyophilized ProQuad®+10 μL diluent). The number of infective particles within the sample (PFU) are referred to herein as particles.

All the Raman spectra of the ProQuad® dilutions were collected with the Renishaw in Via™ Qontorconfocal Raman microscope (Renishaw plc, Wotton-under-Edge, UK). We used a 785-nm excitation laser with 100% (300mW) power and 10 s acquisition time (1 accumulation). The spectral resolution of the spectra was 1 cm⁻¹ , and the spectrum range from 101 to 3200 cm⁻¹ corresponding to 3194 Raman shifts. The samples were focused with a 5× objective of a microscope (LeicaDM2700M), and three replicate Raman spectra were collected for each dilution. The sample volume used for the measurement was 100 μL, and the substrate used for the measurements was a 96-well plate (Corning™ 3635 UV-Transparent Microplates). The experiment was repeated once. The raw Raman spectral data was collected using WIRE 5.5 software. Furthermore, the absorption spectrum for ProQuad® dilutions were collected using the BMG LABTECH, Inc microplate reader (CLARIOstar Plus, SN: 430-2173). The spectrum range was 220 nm to 1000 nm with a spectral resolution of 1 nm wavelength corresponding to 781 wavelengths. The sample volume used for the measurement was 100 μL, and the substrate used for the measurements was a 96-well plate (Corning™ 3635 UV-Transparent Microplates). Three spectral scans for each dilution were collected. The experiment was repeated once. In total, the dataset includes Raman and absorption spectra for 25 different concentration values with three to six replicates for each value, making a total of 116 samples, where 20% of this data is used for testing by 5-fold cross-validation.

### Machine Learning Modeling

Two widely used ML techniques to relate the Raman and absorption spectra to the concentration values were utilized, namely the random forest (RF) and the convolutional neural network (CNN) techniques. Before training, to ensure the reproducibility of the results, all the models are initialized by setting the seed number to zero. To assess the accuracy of predictions, the values of the coefficient of determination (R² scores) were used. Further, to train the models, the 5-fold cross-validation technique is used both for the CNNs and RFs. In this method, the whole data is split into five sections, where the model is trained five times, and each time four sections are used as the training dataset and one section as the testing dataset. The five-fold cross-validation model ensures that all the data points fall into the testing dataset at least once, preventing biased predictions. The Sklearn and Pytorch modules in Python are used for modeling the RFs and CNNs, respectively.

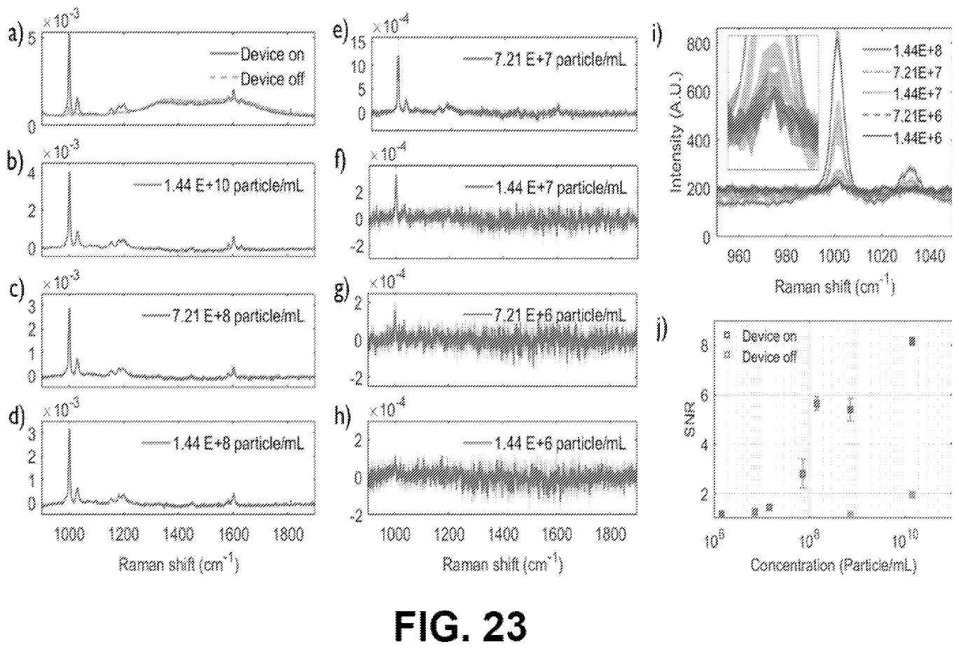

CNN is a supervised machine learning technique that, in the instant case, takes one-dimensional signals as the input and identifies the important parts of the signal, which paves the way for automatic learning of various features and hidden aspects in the signal that are important for the regression. In other words, CNN can capture the spatial and temporal dependencies in the Raman or absorption spectrum. The general architectures of the deep learning models used in this study are similar, i.e., a feed-forward single CNN consisting of four convolutional layers followed by four fully connected layers when either Raman or absorption spectrum are used as the input, as shown in subsection (a) of FIG. 24. However, when it comes to using both the Raman and absorption spectra as the input, two different designs were used. In one design, the Raman and absorption signals were concatenated and fed into a single CNN, as shown in subsection (a) of FIG. 24. In another design, a double CNN is created for feeding the inputs, as demonstrated in subsection (b) of FIG. 24. In the double CNN, the Raman and absorption spectrum are first fed into two separate networks with four convolutional layers and then two fully connected layers. Eventually, the outputs of each network are concatenated and fed into a network with two fully connected layers. In all models, the architecture used for convolutional layers is based on the residual mapping following the deep residual learning method. The presence of residual blocks with shortcut connections between inputs and outputs boosts the training stability and paves the way for having deeper layers.

Furthermore, the kernel size used for all the convolutional layers is three with zero paddings and strides of one. Additionally, all the networks are trained for 6000 epochs (iterations), where further increase in the epochs does not significantly boost the prediction accuracy. The mean squared loss function was used as the criterion for training with the back-propagation techniques, where the stochastic gradient descent with momentum and adaptive learning rate were adopted, where the weight decay and learning rate are set to and 0.1 and 10⁻⁸, respectively. Batch normalization and ReLU activation function are applied consecutively at the end of each convolutional layer, and ReLU function is applied at the end of each fully connected layer. After passing the last ReLU function, the data is mapped into one neuron as the output. The number of channels and neurons are hyperparameters that can be tuned for further accuracy. In the present study, it was found that a maximum of 10 channels and 4000 neurons leads to sufficient accuracy while at the same time avoiding over-fitting.

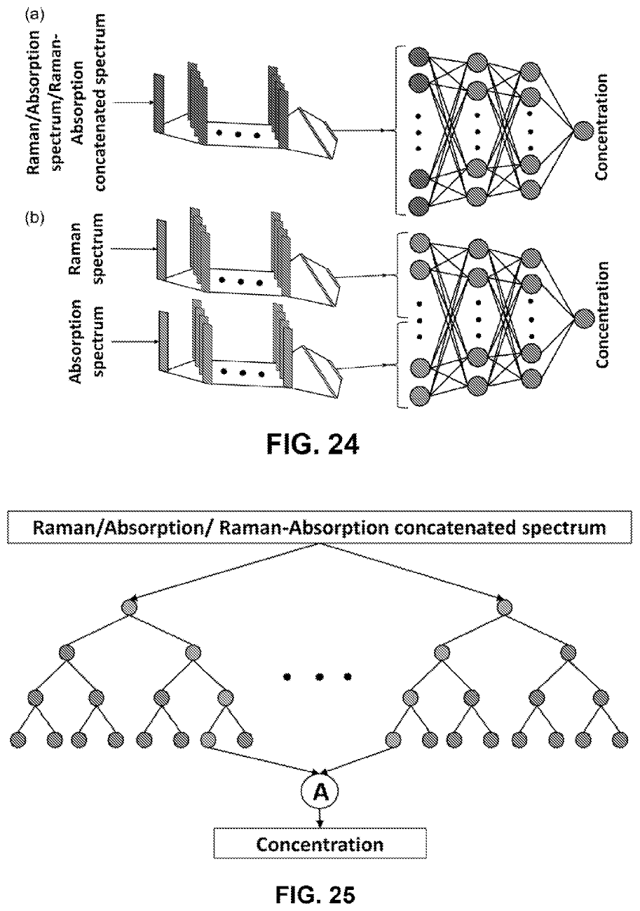

RF regression: RF is a supervised machine learning technique that utilizes the ensemble average of multiple decision trees to make final predictions. Each one of the trees makes its own prediction of the concentration. As shown in FIG. 25, the Raman, absorption, or their concatenated spectrum is used as the input with the concentration as the output. RF is a powerful regression technique that runs efficiently on larger datasets. RFs are generally suitable for making predictions in the training range. Additionally, we use the bootstrapping technique, where we select multiple training samples from the original training sample, and these different samples are used for training each one of these decision trees. Bootstrapping reduces over-fitting chances and stabilizes the network. The squared error criterion in scikit-learn is used to measure the quality of splitting for 100 trees.

### Additional Results

CNN and RF were used as two powerful ML techniques, with different levels of preprocessing to identify the optimum predictions. The algorithms work with the test data generated using 5-fold cross-validation, where each fold can contain points both inside and outside of the training ranges. For CNN, a discussion of whether a single or double CNN works better when both Raman and absorption spectra are used as the input is considered.

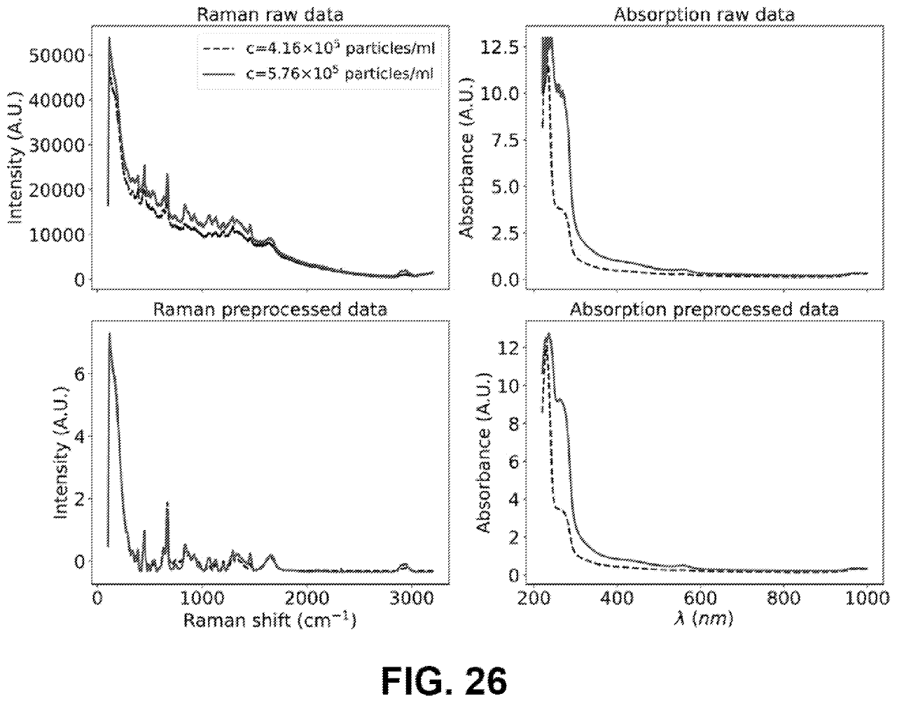

In the present study, CNN models are composed of multiple convolutional layers with the kernel size of 3, where, in each layer, by convolving around the signal, hidden features and patterns are learned. To expedite the learning process and improve the model performance, it is beneficial to preprocess the data before training the models. Thus, baseline corrections were applied and the data was normalized using the standard normal variate method, i.e., subtracting each spectrum by its mean value and dividing by the standard deviation described by Romer et al. Additionally, normalizing the Raman spectrum makes intensities of the Raman and absorption spectrum to be approximately in the same order for further comparison. No baseline correction or normalization is required for the absorption spectrum since the difference between maximum and minimum values is relatively low. Additionally, normalizing the absorption data led to no significant boost in prediction accuracy. Finally, the Raman and absorption signals are smoothened using the Savitzky-Golay (SG) filter. FIG. 26 demonstrates the Raman and absorption spectra before and after preprocessing for two different concentrations. In addition to normalization and applying filters, some studies trim the Raman spectrum to obtain the spectral range of interest. In the current study, mp significant gain in the prediction accuracy was observed when the Raman or absorption spectrum is trimmed, as shown, for example, for the RF method described below. Additionally, an analysis of how the predictions change with the subtraction of the control spectrum of solvent was performed as described below, where a reduction in the accuracy with the subtraction of the control spectrum was noticed, and as such subtraction of the control spectrum step was excluded from preprocessing steps.

The R² coefficients for the values of the 5-fold predictions for both RF and CNN are listed in Table 1, below:

TABLE 1
The R² values of 5-fold cross-validation for the prediction of concentration
for given Raman, absorption, and Raman-Absorption concatenated spectra.

| | | | Raman- | | | | Raman- |
|---|---|---|---|---|---|---|---|
| Fold | Absorption | Raman | Absorption | Concatenated R-A | Absorption | Raman | Absorption |
| 1 | 0.975 | 0.976 | 0.981 | 0.992 | 0.977 | 0.974 | 0.988 |
| 2 | 0.948 | 0.973 | 0.980 | 0.974 | 0.974 | 0.983 | 0.995 |
| 3 | 0.942 | 0.980 | 0.977 | 0.992 | 0.964 | 0.979 | 0.991 |
| 4 | 0.835 | 0.964 | 0.931 | 0.946 | 0.881 | 0.953 | 0.981 |
| 5 | 0.952 | 0.984 | 0.980 | 0.991 | 0.989 | 0.982 | 0.990 |
| Ave | 0.930 | 0.975 | 0.969 | 0.979 | 0.955 | 0.974 | 0.989 |

The average R² score for all the predictions is above 90%. However, the prediction accuracy is higher when the concatenated Raman-absorption spectrum is used for RF and CNN compared to the predictions based on either Raman or absorption spectrum. Furthermore, the prediction accuracy is slightly higher for RF compared to CNN in the hyper-parameter space that was studied. However, both RF and CNN lead to predictions with R² values as high as 98% when the joint Raman-absorption data is used. Additionally, it is noted that the single CNN demonstrates higher prediction accuracy compared to the double CNN, which may be attributed to the low predictability of the absorption spectrum compared to the Raman spectrum, as the prediction accuracy is higher when only Raman is used compared to when only absorption data is used.

Furthermore, a comparison between RF and support vector machine (SVM) methods was made, where it is noted that RF predictions are slightly more accurate than SVM. Several machine learning (ML) algorithms can be used for making predictions using Raman and absorption spectra. Table 2 demonstrates the R² coefficients for the values of the 5-fold predictions for the SVM method. The results are very similar to the values listed for the RF method for the Raman and concatenated Raman-absorption spectrum. However, for the absorption spectrum, it is noted that the RF predictions are more accurate than SVM. Tables 3, 4, and 5 provide related data.

TABLE 2
The R² values of 5-fold cross-validation for the prediction of concentration
for the Raman, absorption, and concatenated Raman-absorption spectrum
using the SVM methods.
SVM

| Fold | Absorption | Raman | Raman-absorption |
|---|---|---|---|
| 1 | 0.664 | 0.980 | 0.991 |
| 2 | 0.698 | 0.984 | 0.990 |
| 3 | 0.777 | 0.981 | 0.989 |
| 4 | 0.601 | 0.968 | 0.980 |
| 5 | 0.621 | 0.984 | 0.993 |
| Ave | 0.672 | 0.979 | 0.988 |

| | RF | | | SVM | | |
|---|---|---|---|---|---|---|
| Fold | Absorption | Raman | Raman-absorption | Absorption | Raman | Raman-Absorption |
| 1 | 0.958 | 0.926 | 0.933 | 0.664 | 0.731 | 0.701 |
| 2 | 0.972 | 0.935 | 0.960 | 0.698 | 0.746 | 0.790 |
| 3 | 0.949 | 0.951 | 0.940 | 0.777 | 0.623 | 0.578 |
| 4 | 0.873 | 0.914 | 0.973 | 0.601 | 0.758 | 0.793 |
| 5 | 0.973 | 0.957 | 0.973 | 0.621 | 0.695 | 0.538 |
| Ave | 0.945 | 0.937 | 0.956 | 0.672 | 0.710 | 0.680 |

Table 3 shows the R² values of 5-fold cross-validation for the prediction of concentration for the Raman, absorption, and concatenated Raman-absorption spectrum using PCA.

Regarding Table 3, it is noted that the background noise can effect the Raman and absorption spectra, particularly at low Raman shifts and wavelengths. As a result, the initial parts of the Raman (Raman shift <300 cm⁻¹) and the absorption spectrum (λ<250 nm) were removed. As noted in Table 4 below, the prediction accuracies do not change significantly with trimming. Therefore, the entire spectra was used for prediction. An advantage of using ML technique is that these techniques automatically detect which part of the signal is important.

TABLE 4
The R² values of 5-fold cross-validation for the prediction of
concentration for the trimmed Raman, absorption, and concatenated
Raman-absorption spectrum using the RF method.
RF

| Fold | Absorption | Raman | Raman-absorption |
|---|---|---|---|
| 1 | 0.958 | 0.971 | 0.987 |
| 2 | 0.972 | 0.982 | 0.991 |
| 3 | 0.949 | 0.967 | 0.975 |
| 4 | 0.873 | 0.961 | 0.955 |
| 5 | 0.973 | 0.980 | 0.988 |
| Ave | 0.945 | 0.972 | 0.979 |

Regarding Table 4, the control spectrum of the solvent (sterile water) was not subtracted from the Raman and absorption spectrum to minimize the amount of preprocessing. Here it is demonstrated how the prediction accuracies change if the control data is subtracted from all of the spectrums. As evident, the presence of viral particles induces noticeable changes at most Raman shifts. Further, the absorption signal at all wavelengths is different when viral particles are introduced.

In addition, the spectrum with the water data subtracted was presented as well. Table 5 demonstrates R² values for predictions of the RF method using the spectrum with water data subtracted. It is noted that the R² values decrease with the subtraction of water data compared to the values presented in Table 4. As such, the subtraction of the water spectrum step was excluded in the preprocessing.

TABLE 5
The R² values of 5-fold cross-validation for the prediction of concentration
for the trimmed Raman, absorption, and concatenated Raman-absorption
spectrum using the RF method with control data being subtracted.
RF

| Fold | Absorption | Raman | Raman-absorption |
|---|---|---|---|
| 1 | 0.975 | 0.920 | 0.981 |
| 2 | 0.982 | 0.983 | 0.991 |
| 3 | 0.971 | 0.931 | 0.948 |
| 4 | 0.882 | 0.866 | 0.917 |
| 5 | 0.989 | 0.947 | 0.974 |
| Ave | 0.959 | 0.972 | 0.962 |

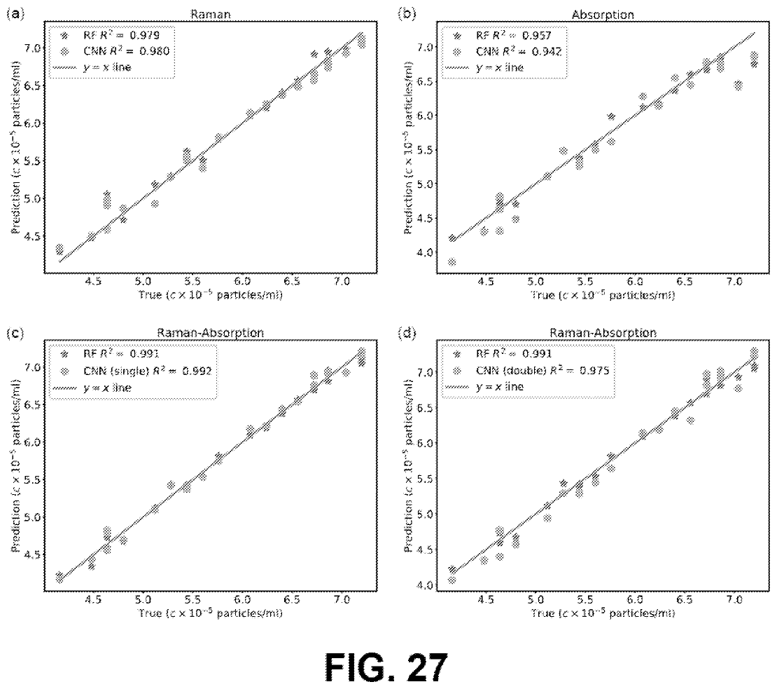

Additionally, a visual demonstration of how the predictions of CNN and RF vary for the testing dataset in one of the folds in the 5-fold dataset was performed. As demonstrated in FIG. 27, prediction values based on the Raman spectrum are more in line with the actual values as opposed to the absorption spectrum, where the average R² coefficient is lower. This difference can be attributed to the larger size of the Raman signal and, therefore, larger regions of dissimilarity corresponding to different concentrations, which make Raman spectra more distinguishable from each other. Further, the use of joint Raman-absorption spectra boosts the prediction accuracy compared to the case when only Raman spectra are used.

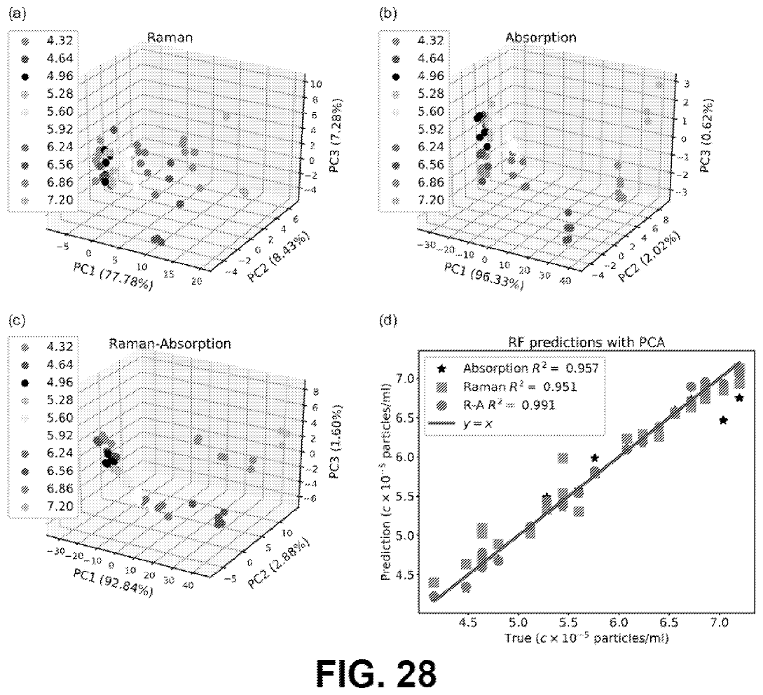

The differences between the prediction accuracy of the Raman and the absorption spectra can further be understood through the principal component analysis (PCA). PCA was used to reduce the dimensionality of the Raman, absorption, and concatenated Raman-absorption spectra to 4, where the original size of the Raman and absorption spectra are 3194 and 781. FIG. 28 demonstrates how principal coordinate (PC) values differ at different concentration values. The distinction between PCA points at different concentrations is more evident for the Raman-absorption spectrum as compared to the Raman or absorption spectrum. Additionally, it was noticed that for most cases, not only the prediction accuracy does not increase by conducting PCA, but also for the Raman and Raman-absorption data, the average R² values slightly decrease when subsection (d) of FIG. 28 and FIG. 27 are compared. Therefore, for the current dataset, dimensionality reduction does not improve the prediction accuracy.

### Discussion

In the studies noted above, the possibility of using absorption, Raman, and joint Raman-absorption spectrum to determine the concentration of the samples containing viral particles was investigated. RF and CNN, as two different machine learning algorithms, were utilized for making predictions, and the prediction accuracy was monitored using 5-fold cross-validation. It was demonstrated that with sufficient preprocessing, both the Raman and absorption spectra could be used to create a surrogate to predict the values of concentration. In most cases, the Raman spectrum leads to more accurate predictions compared to the absorption spectrum. Moreover, concatenating Raman and absorption spectra improves the prediction accuracy both for RF and CNN. Furthermore, PCA analysis sheds light on the advantage of joint spectra over single usage of Raman or absorption spectrum as the points corresponding to different concentrations are further separated. It was also demonstrated that the joint utilization of the Raman and absorption spectra paves the way for the real-time measurements of the concentration of the viral particles in well plates, which can be extended to different static and dynamics settings, such as microfluidic devices with different flow conditions.

Some limitations of this study can be listed as follows. a) the predictions, in general, work well when the unknown concentration values lie in the range of training datasets. Given that here the focus was on relatively large concentration values (>4×10⁵/ml), the predictions for the low concentration values (<<4×10⁵/ml) are not reliable. b) the predictions are valid only for ProQuad® samples. Further training data points corresponding to different types of viral particles are required to extend the applicability of the current method.

The present disclosure demonstrates that Raman spectroscopy is a promising process analytical tool in the future of continuous manufacturing of biologics. As previously discussed, the main bottleneck in continuous manufacturing is the lack of efficient PAT tools to satisfy the needs of analytical monitoring. As discussed herein, three different acoustic devices were generated, and Raman peaks were used as a marker for tracking attenuated CMV and different vaccine samples. The incorporation of artificial intelligence and machine learning approaches along with the specific Raman peaks and absorption measurements provided more detailed information with the analysis of complex viral particles.

While various embodiments of devices, systems, and methods of using and making the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A device for analyzing microparticles and nanoparticles, comprising:
an elongated capillary tube having a square cross-section; and
a substrate configured to receive the elongated capillary tube thereon;
wherein the elongated capillary tube is affixed to the substrate using an adhesive.

2. The device of claim 1, further comprising:
a first plastic tube positioned relative to and about a first end of the elongated capillary tube and coupled thereto using the adhesive.

3. (canceled)

4. The device of claim 3, further comprising:
a second plastic tube positioned relative to and about a second end of the elongated capillary tube and coupled thereto using the adhesive; and
a pump in fluid communication with one of the first plastic tube and the second plastic tube, the pump configured to pump a fluid having a virus therein through the first plastic tube or the second plastic tube and into the elongated capillary tube.

5. (canceled)

6. The device of claim 1, wherein the substrate comprises a lithium niobate (LiNBO₃) substrate.

7. The device of claim 6, wherein the substrate comprises an interdigital transducer (IDT) electrode positioned thereon or defined therein.

8. The device of claim 7, wherein a surface acoustic wave (SAW) is activated by a radio signal from a power supply connected to the IDT.

9. The device of claim 8, wherein the SAW produced by the IDT travels through the LiNBO₃ substrate and a thin layer of UV epoxy to reach the elongated capillary tube.

10. The device of claim 9, wherein the SAW causes the bottom of the elongated capillary tube to vibrate, which generates an acoustic force that acts on the fluid and microparticles and nanoparticles inside.

11. (canceled)

12. The device of claim 6, prepared by:
applying a photoresist onto the LiNBO₃ substrate;
applying ultraviolet light to a portion of the photoresist;
developing the photoresist to form a void therein; and
depositing metal onto the photoresist and into the void produced by the step of developing the photoresist.

13. The device of claim 12, further prepared by:
removing the photoresist from the LiNBO₃ substrate, effectively forming an interdigital transducer (IDT); and
applying a conducting epoxy to the metal deposited onto the LiNBO₃ substrate and applying a clear ultraviolet (UV) epoxy to a portion of the LiNBO₃ substrate.

14. The device of claim 13, further prepared by:
affixing the elongated capillary tube to the LiNBO₃ substrate upon the UV epoxy.

15. The device of claim 12, wherein the deposited metal comprises titanium and gold.

16. The device of claim 15, wherein the titanium is deposited onto the LiNBO₃ substrate, and wherein the gold is deposited onto the titanium.

17. (canceled)

18. (canceled)

19. The device of claim 7, wherein the IDT comprises two opposing vertical bars, each vertical bar of the opposing vertical bars having alternating horizontal fingers extending therefrom.

20. (canceled)

21. (canceled)

22. The device of claim 7, forming part of a system, the system further comprising:
a function generator in communication with a power supply, the power supply effectively coupled to the IDT; and
a Raman analyzer positioned relative to the elongated capillary tube.

23. The system of claim 22, configured so that wherein when power is supplied from the power supply to the IDT, the IDT is configured to generate a surface acoustic wave (SAW) that travels through the LiNBO₃ substrate to the elongated capillary tube, causing the elongated capillary tube to vibrate.

24. (canceled)

25. (canceled)

26. A system for analyzing microparticles and nanoparticles, comprising:
the device of claim 4; and
a Raman microscope having a lens directed at the device;
wherein when the fluid having the virus therein is pumped into the elongated capillary tube, the Raman microscope is used in connection with an excitation laser to obtain Raman spectra in connection with the virus.

27. A system for analyzing microparticles and nanoparticles, comprising:
the device of claim 14; and
a generator configured to generate an electrical signal for actuation with different frequencies; and
an amplifier configured to amplify the electrical signal.

28. (canceled)

29. (canceled)

30. (canceled)

31. A method for analyzing microparticles and nanoparticles, comprising the steps of:
positioning the device of claim 4 relative to a Raman spectroscopy device; and
operating the Raman spectroscopy device to obtain data relative to the virus present within the capillary tube.

32. A method for analyzing microparticles and nanoparticles, comprising the steps of:
positioning the device of claim 7 relative to a Raman spectroscopy device; and
operating a generator coupled to an amplifier and configured to generate an electrical signal upon the IDT;
wherein a surface acoustic wave (SAW) generated by the IDT due to the electrical signal travels through the LiNBO₃ substrate and a thin layer of UV epoxy to reach the elongated capillary tube.

33. (canceled)

34. (canceled)
